(19) United States
(12) Patent Application Publication
(10) Pub. No.: US 2025/0325288 A1
(43) Pub. Date: Oct. 23, 2025

(54) Shaped Inflatable Device

(71) Applicant: Motaz Shaher, Fort Wayne, IN (US)

(72) Inventor: Motaz Shaher, Fort Wayne, IN (US)

(21) Appl. No.: 19/065,269

(22) Filed: Feb. 27, 2025

Related U.S. Application Data
(60) Provisional application No. 63/559,582, filed on Feb. 29, 2024.

(51) Int. Cl. A61B 17/22; A61B 17/12

(52) U.S. Cl. CPC A61B 17/22; A61B 2017/1205; A61B 2017/22001; A61B 2017/22048; A61B 2017/22055; A61B 2017/22065

(57) ABSTRACT

The present invention relates to inflatable devices such as coronary and peripheral angioplasty devices, which can achieve better expansion of blockages in various types of blood vessels, and expansion of other cardiac and non-chambers and/or channels in the body. Further, the present invention can also be used in other inflatable devices that can be used to open other blockages in cardiology, urology, gastrointestinal, biliary ducts, non-medical blockages, etc. present invention provides a shaped inflatable apparatus, system, and/or device for treating pathological narrowing of and/or expansion of blockages in fluid-carrying conduits such as blood vessels in a body of an animal, the inflatable apparatus, system, and/or device comprises an inflatable chamber, wherein the inflatable chamber provides for better expansion of blockages in various types of blood vessels, and expansion of other cardiac and non-chambers and/or channels in the body, wherein the inflatable chamber is a non-cylindrical shape or non-circular in cross-section.

### PRIORITY

This application claims priority for the U.S. provisional application Ser. No. 63/559,582 filed on Feb. 29, 2024.

### FIELD OF THE INVENTION

The present invention generally relates to better inflatable apparatus, systems, and devices, as well as methods for use thereof. More specifically, it relates to shaped inflatable apparatus, systems, and devices, as well as methods of use thereof to combat blockages in fluid-carrying conduits in cardiology, urology, gastrointestinal, biliary ducts, as well as non-medical blockages, etc.

### INCORPORATION BY REFERENCE

All publications, patents, and patent applications mentioned in this specification are herein incorporated by reference in their entirety to the same extent as if each publication or patent application was specifically and individually indicated to be incorporated by reference.

### BACKGROUND OF THE INVENTION

Coronary artery disease is the most common cause of death in the US. It is usually associated with progressive narrowing of the coronary arteries because of cholesterol and calcium plaque buildup in the vessel wall. Plaque build-up is usually not circumferential and mostly patchy.

As provided herein, FIG. 1 illustrates a representative example of severe coronary artery obstruction that is traditionally treated in the art by dilating the affected vessel, usually by inflating a balloon mounted on a special catheter across the narrowed section of the vessel, followed by placement of metallic scaffolding (stent) to prevent the vessel from collapsing and narrowing again after deflating the balloon and withdrawing it.

Most current balloons used in angioplasty devices are elongated and cylindrical in shape, with a circular cross-sectional profile due to the uniform outward expansion of the balloon when inflated, which causes an almost uniform distribution of the force being applied to the blockage within the vessel's wall, when the balloon is inflated, thus inflating the balloon into a narrowed section of the vessel results in the same force being applied to the normal portion as well as the diseased portion of the vessel wall. Provided herein is a representative example of a standard design of a conventional rapid exchange balloon angioplasty catheter is shown in FIG. 2, which consists of a balloon that is mounted around the shaft of the delivery catheter with a central or off-center channel used for advancement of a guide wire (Reference: Amstutz et al. BioMedical Engineering OnLine (2023) 22:94). There is another inflation channel along the length of the catheter which is used to inflate the balloon.

Further shown herein in FIG. 3 are mechanisms of opening a narrowed vessel device and increasing the luminal size of the vessel using a Balloon angioplasty usually by rupturing atherosclerotic plaque, dissection of the plaque, compression of the plaque, and/or vessel wall expansion.

Moreover, provided herein in FIG. 4 is a currently used balloon angioplasty device example highlighting how these devices can result in an unnecessary injury to the normal segments of the vessel because a higher force is needed to expand the diseased segment, which will also be applied to the normal segment. Besides, parts of the balloon may not expand or minimally expand if the adjacent segment of the vessel is very stiff due to calcification or fibrosis.

For example, U.S. Pat. No. 905,0182B2 provides illustrations of existing stent/graft assemblies that includes a tubular graft connected in substantially end-to-end relationship with a generally tubular stent. Further, it provides that endovascular stent/graft assemblies may exhibit some flexibility. Thus, a conventional cylindrical endovascular stent/graft assembly can be biased into a noncylindrical curved shape that conforms to the shape of the neck adjacent the aneurysm. However, the prior art teaches that an initially cylindrical stent/graft assembly with a linear axis of symmetry that is biased into a curved noncylindrical shape will exhibit internal resiliency that will tend to return the stent/graft assembly back to an unbiased cylindrical configuration.

Another example, U.S. Pat. No. 6,605,107B1 provides other examples of existing prostheses and methods for their endoluminal placement within body lumens, particularly blood vessels. In one embodiment as provided therein, the prosthesis consists of a plurality of radially expansible rings having, a plurality of beams connecting axially remote points on adjacent rings, and a plurality of expansion joints connecting axially proximate points on adjacent rings. Further, it mentions that a prosthesis can be mounted on an inflation member such as a balloon which can shape the expanded prosthesis into a noncylindrical shape. For example, the prosthesis may be expanded into a tapered configuration to provide improved fit and scaffolding in body lumens which have conical configurations. The ability of the inflation member, such as a noncylindrical balloon, to expand a prosthesis into a shape that better conforms to the luminal geometry may also be enhanced by having unit segments of various radial stiffnesses, such as having a stiffness gradient from one segment to the next, or other nonuniform stiffnesses along the length of the prosthesis.

Thus, the current conventional inflatable devices such as the balloon angioplasty designs and devices in use have several limitations, including the fact that in the process of dilating a narrowed vessel, a higher force has to be applied to the normal portion of the blood vessel as well as the diseased portion, which can result in a vessel wall dissection and a possible tear and perforation of the wall as has been illustrated in a representative example in FIG. 5 as disclosed herein. Additionally, the current devices are unable to apply higher force that is necessary to expand a calcified plaque within the vessel wall because of the fear that this may result in injury to the normal segments of the blood vessel. Also, nonselective and/or diffuse application of the balloon inflation's force to the calcified plaque can lower the chance of fracturing the plaque, which is usually needed to expand the narrowed segment of the vessel as shown also in FIG. 5. Uniform and diffuse application of force by inflating the balloon does not distribute the force specifically to the diseased portion of the vessel, which is needed to expand the blood vessel. In calcified vessels, fissures and cracks need to be created within the calcified plaque to enable the expansion of the vessel narrowing, this is difficult to achieve due to the uniform distribution of the force being applied by the inflated balloon, which can result in suboptimal or lack of expansion of the narrowed vessels.

The main reasons for the traditional use of uniform cylindrical (circular profile) elongated balloon angioplasty are familiarity and ease of use, as the medical practitioner needs to inflate the cylindrical balloon without the need for precise knowledge of the location of the plaque, or the orientation of the atherosclerotic plaque inside the blood vessel wall.

However, there is a need for a new design of the inflatable chamber part for better expansion of blockages in various types of blood vessels, and expansion of other cardiac and non-cardiac chambers and/or channels in the body with the use of non-cylindrical (non-circular in cross-section of the device) shape to enable more precise application of the force and energy needed to expand a narrowing in the blood vessel, aperture, chamber, etc. to open blockages in cardiovascular system, urology, gastrointestinal, pulmonary, biliary ducts, as well as non-medical blockages, etc.

The present invention addresses this need and problem by providing an inflatable device with new designs of the inflatable chamber part as disclosed in the present invention to achieve a better distribution of the forces being applied and allows the use of a lower nominal pressure of the inflatable device to reduce acute complications associated with the conventional devices currently in use in the art.

### SUMMARY OF THE INVENTION

The following listing of embodiments is a nonlimiting statement of various aspects of the invention. Other aspects and variations will be evident in light of the entire disclosure.

The present invention provides better inflatable devices such as coronary and peripheral angioplasty devices, which can achieve better expansion of blockages in various types of blood vessels, and expansion of other cardiac and non-cardiac chambers and/or channels in the body. The present invention as disclosed herein can be used in other inflatable devices that can be used to open other blockages in cardiology, urology, gastrointestinal, biliary ducts, as well as non-medical blockages, etc.

An aspect of the present invention provides a shaped inflatable apparatus, system, and/or device for treating pathological narrowing of and/or expansion of blockages in fluid-carrying conduits such as blood vessels in a body of an animal, the inflatable apparatus, system, and/or device comprises an inflatable chamber, wherein the inflatable chamber provides for better expansion of blockages in various types of blood vessels, and expansion of other cardiac and non-chambers and/or channels in the body, wherein the inflatable chamber is non-cylindrical shape or non-circular in cross section of the apparatus, system, and/or device, wherein the animal is a mammal, and wherein the mammal is a human.

An aspect of the present invention provides a shaped inflatable apparatus, system, and/or device for treating pathological narrowing of and/or expansion of blockages in fluid-carrying conduits such as blood vessels in a body of an animal, the inflatable apparatus, system, and/or device comprises a shaft; an inflation channel(s); one or more guide-wire channel(s); one or more stiffening wire; an inflation port(s); one or more guide-wire port; and a shaft tip; and optionally it further comprises a varying combination of parts selected from a group comprising one or more protrusions, wherein the protrusions are bulging of an inflation chamber periphery, with the wall of the protrusion that is contiguous with the inflatable chamber's wall, typically a main inflatable chamber to allow same time inflation; one or more side inflation chambers, wherein the side inflatable chambers are the ones with a wall that partially or completely separates them from the main inflatable chamber, and optionally they have their own inflatable channel, and/or they share an inflatable channel with a main inflatable chamber; one or more shafts made from flexible material, wherein the shafts instead of being the typical cylindrical in shape and itself is typically non-inflatable.

Other objects, features, and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The accompanying drawings are included to provide a further understanding of the invention and are incorporated in and constitute a part of the present invention and, together with the description, serve to explain the principle of the invention.

In the drawings,

FIG. 1 illustrates a representative illustration of cross-section of a blood vessel showing the patchy nature of atherosclerotic plaque inside the blood vessel wall (2) affecting only part of the circumference of the blood vessel. Identified in the illustration are the blood vessel wall (1), atherosclerotic plaque (2) inside the blood vessel wall, lumen of the blood vessel (3), and balloon angioplasty device (4).

FIG. 2 illustrates a representative illustration of a standard balloon angioplasty (RX PTCA Balloon Catheter), identified in the illustration are a metallic hypotube (1), transition between proximal and distal part (2), distal part of the catheter including the inner shaft for the guidewire and the outer shaft for inflating the balloon (3), (folded) balloon with radio-opaque markers (4), a tip (5), Rx-Port (6), a stiffening wire (7), and an inflation port 8).

FIG. 3 illustrates a representative illustration of a cross-section of a blood vessel showing that the balloon angioplasty device may have to fracture the atherosclerotic plaque (4) to achieve expansion of the vessel lumen, especially if the atherosclerotic plaque (3) is calcified thus limiting the expansion of the balloon angioplasty device (5). Also identified in the illustration are the blood vessel wall (1), atherosclerotic plaque (3), inside the blood vessel wall, lumen of the blood vessel (5), and balloon angioplasty device (2).

FIG. 4 illustrates a representative illustration of a cross-section of a blood vessel showing a calcified fibrotic atherosclerotic plaque inside the blood vessel wall (2) that may limit the expansion (inflation) of the balloon angioplasty device (5). Also identified in the illustration are the blood vessel wall (1), atherosclerotic plaque (2) inside the blood vessel wall, lumen of the blood vessel (3), and a conventional balloon angioplasty device (4).

FIG. 5 illustrates a representative illustration of a cross-section of a blood vessel showing that during inflation of a standard balloon angioplasty device (1), the normal segments of the blood vessel (2) are being exposed to high inflation pressures (3), while trying to expand a calcified fibrotic atherosclerotic plaque (4) inside the blood vessel wall to induced rupture in the plaque (5) that may limit the expansion (inflation) of the balloon angioplasty device (1). Thus, most of the inflation pressure being applied to the vessel is unnecessary except for a small area within the balloon (6).

FIG. 6 illustrates in panel A, a representative illustration of a standard balloon angioplasty device from the longitudinal axis view, it shows a cylindrical balloon inflated with a material. Identified in the illustration are the balloon (1), inflation channel (2), guidewire channel (3), tip of the device (4), and the shaft of the device (5). Further, in panel B of FIG. 6 it illustrates a representative illustration of the same representative device in cross sectional view at the level of the balloon revealing that the standard inflated balloon is circular in shape and identified are the balloon (1), and a guidewire channel (2).

FIG. 7 illustrates an embodiment of the present invention and illustrates a representative example of a new balloon angioplasty device based on the new invention from the long axis view, it shows that the standard cylindrical inflated balloon is replaced with a noncylindrical balloon, but the other components of the device are similar. Identified in the illustration are the inflatable chamber (1), inflation channel (2), guidewire channel (3), tip of the device (4), and shaft of the device (5).

FIG. 8 illustrates an embodiment of the present invention and illustrates a representative example of a shaped inflatable device and its components that may form the inflatable device. Identified in the illustration are the representative device's shaft (1), non-cylindrical inflatable chamber (2), inflation channel (3), component to secure the device in place to the patient's skin (4), guide-wire channel (5), inflation port (6), device's tip (7), guidewire port (8), span of catheter's shaft 9, span of the inflatable chamber 10, span of the tip of the catheter (11). The illustrated span catheter as well as the span of the tip can be variable based on the indication for the procedure.

FIG. 9A and 9B illustrate embodiments of the present invention and illustrates first in panel A, a representative example of one of the embodiments of the device showing a longitudinal axis view of an angioplasty device with an oval shaped balloon, as viewed from the Z-axis of the device, (Z-axis is the longitudinal axis of the device's shaft). Inflatable chamber (1), guidewire channel (2), inflation channel (3), maximal width of the balloon (4), length of the balloon (5). Y-axis of the catheter is perpendicular to the Z-axis, while X-axis (not shown here) is perpendicular to the Z- and Y-axis. In panel B, it illustrates one of the embodiments of the device with a cross-sectional view of an angioplasty device with an oval shaped balloon, as viewed from the X- and Y-axis of the device, X- and Y-axis are the perpendicular axis to the device's shaft and/or its Z-axis, and identified in the illustration are the inflatable chamber (1), and the device's shaft (2). In panel C, it illustrates one of the embodiments of an inflated device showing a cross-sectional long axis view. In this embodiment, the device has a non-cylindrical inflatable chamber (1), but with different orientation of the inflatable chamber when compared to the illustration in the preceding panel A and panel B. The balloon is asymmetric in relation to the device's shaft Z-axis, so that the device's shaft axis, center-line of the device's shaft, is not in the center of the inflatable chamber, thus resulting in inflatable chamber width (6) that is different than the width of the inflatable chamber from the opposite side (7). Also identified in the illustration are the guidewire channel (2), inflation channel (3), maximal width of the balloon (6, 7), length of the inflatable chamber (5). In panel D, it illustrates one of the embodiments of an inflated device showing a cross-sectional short axis view. In this embodiment, the device has an oval asymmetric inflatable chamber (1). The inflatable chamber (1) is asymmetric in relation to the device's shaft or the guidewire channel's X- and Y-axis. In this embodiment, the device's shaft axis is not in the center of the inflatable chamber, thus resulting in variable inflatable chamber width (3, 4, 5, 6) as measured from the guidewire channel (2). In panel E, it illustrates one of the embodiments of an inflated device showing a cross-sectional short axis view. In this embodiment, the device has an oval asymmetric balloon (1). Further identified in the illustration are the guidewire channel (2) is adjacent, and can be in some other embodiments, attached to the wall of the inflatable chamber, thus resulting in the discrepant width of the inflatable chamber as measured from the guidewire channel (3, 4).

FIG. 10 illustrates an embodiment of the present invention and illustrates a representative example of one of the embodiments of an inflated device showing a cross-sectional long axis view. In this embodiment, the device has an angulated balloon (1), when compared to the device's shaft and guidewire channel (4). The angle between the inflated balloon in relation to the device's shaft or the guidewire channel (3) can range from 0-359 degrees, and also identified is the inflation channel (2).

FIG. 11 illustrates an embodiment of the present invention and illustrates in panel A, a representative example of one of the embodiments of an inflated device showing a cross-sectional long axis view. In this embodiment, the device has two main inflatable chambers (3, 5), which are connected through openings (1, 4), in the inflatable channels (6, 8). Main inflatable chamber (5) shares a wall (10) with the catheter's shaft (7). In this embodiment, the main inflation chamber is fused in an air-tight bonding to the catheter's shaft and the inflation channel. Also identified in this illustration are guidewire channel (9), inflatable chamber that has a fluid and/or gas tight connection (11) to the catheter's shaft, and the tip of the catheter (2). In panel B of FIG. 11, it illustrates one of the embodiments of an inflated device showing a cross-sectional short axis view. In this embodiment, the device has two main inflatable chambers (2, 7), which are connected through openings (3, 6) in a common inflatable channel (1). Also identified in the illustration are main inflatable chamber (2) that shares a wall (9) with the catheter's shaft (4), and a guidewire channel (5).

FIG. 12 illustrates an embodiment of the present invention and illustrates in panel A, a representative example of one of the embodiments of an inflated device showing a cross-sectional short axis view. In this embodiment, the device has main inflatable chambers (6, 5, 2, 10, 8), with each main inflatable chamber having its own inflatable channel (7, 9, 3, 4, 11), respectively. Also identified in the illustration are a guidewire channel 1, main Inflatable chambers that may have different sizes and/or shapes, and which follow a certain pattern in relation to the device's shaft (devices shaft is represented by the inflatable channels and guidewire channel). In this embodiment, in relation to the catheter's shaft (inflation channels and guidewire channel), the main inflatable chambers (6) and (8) are small and similar in shape and size when inflated, with two additional inflatable chambers (5) and (10) that are similar in shape and size when inflated, but are different in shape and size than (6, 8), and there is an additional larger inflatable chamber (2) on the other side. In panel B, illustration of one of the embodiments of an inflated device showing a cross-sectional short axis view. In this embodiment, the device is the same device as the one in Panel A of FIG. 12, but in this illustration, it is illustrated that the device at the catheter's shaft level, it shows main inflatable channel (7, 9, 3, 4, 11) passing inside the catheter's shaft (12) and also shown is a guidewire channel (1).

FIG. 13 illustrates an embodiment of the present invention and illustrates in panel A, a representative example of one of the embodiments of an inflated device showing a cross-sectional short axis view. In this embodiment, the device has main inflatable chambers (3, 4, 5), with a common inflatable channel (6), that is used to fill all of these chambers and also identified is a guidewire channel (7). All of the main inflatable chambers are encased in a larger inflatable chamber (1), which has its own inflation channel (2). In other embodiments, the encasing chamber (1) may share inflation channel with the main inflatable chambers. In some other embodiments, the larger chamber (1) may have no cavity or inflation channel, and may be just made from flexible material that is not fillable.

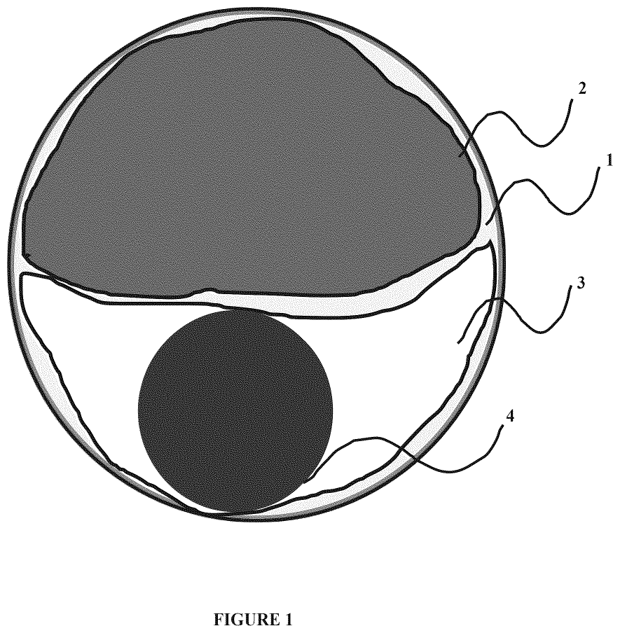

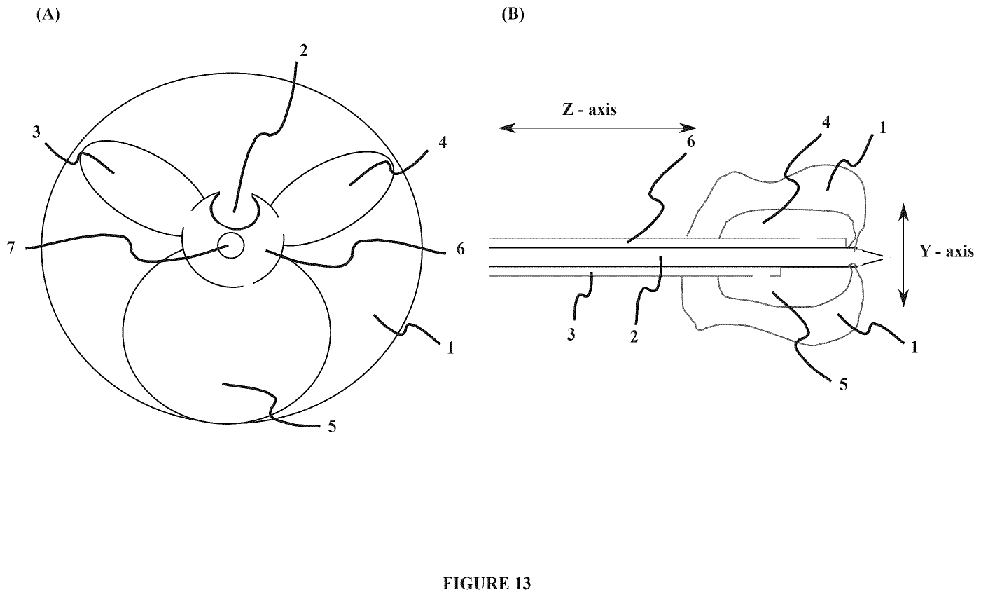

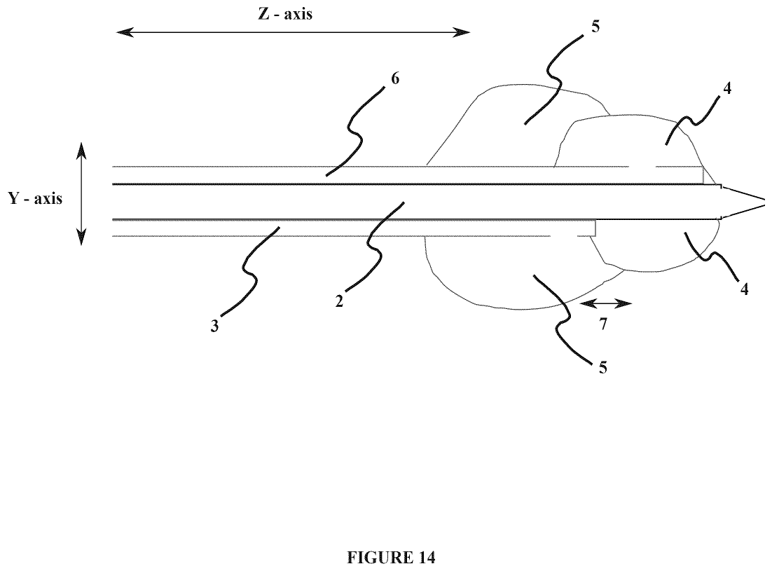

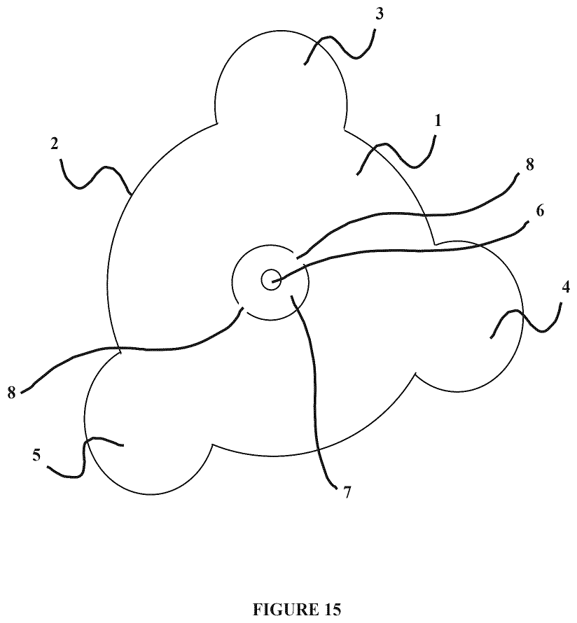

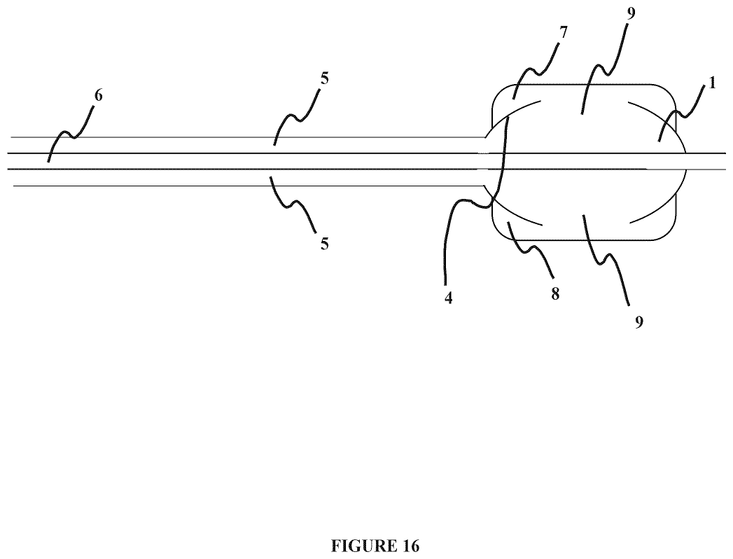

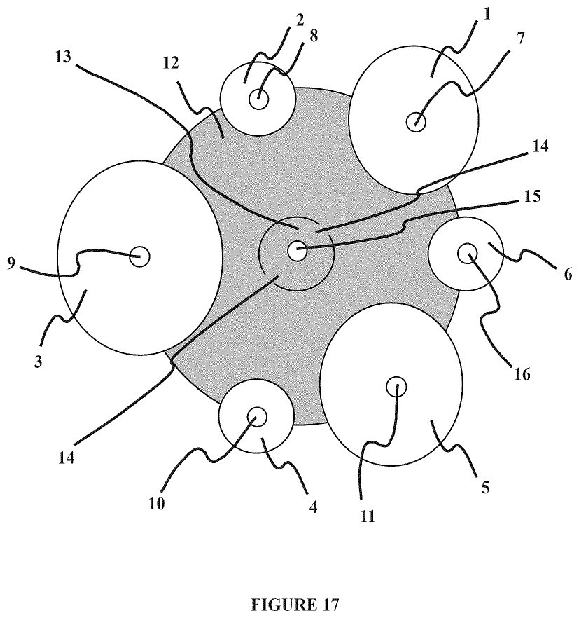

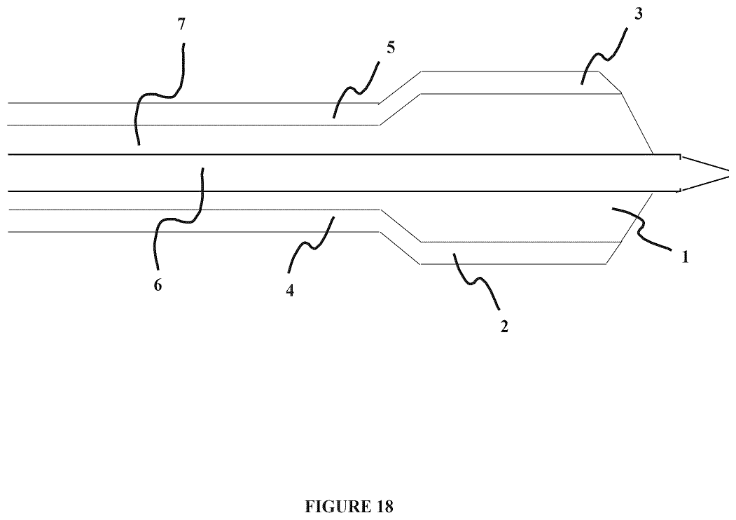

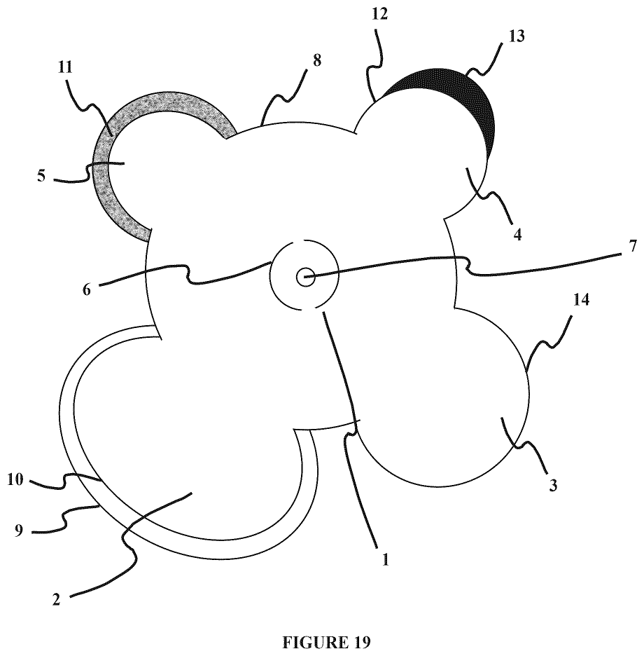

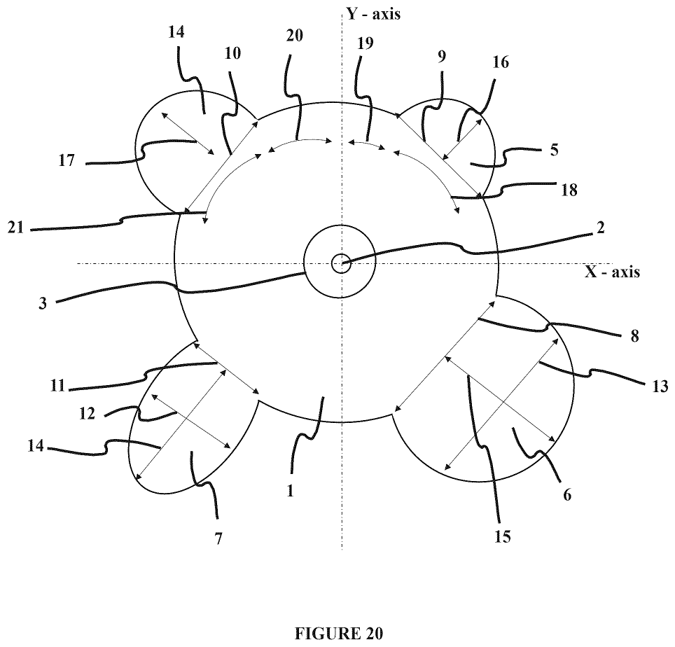

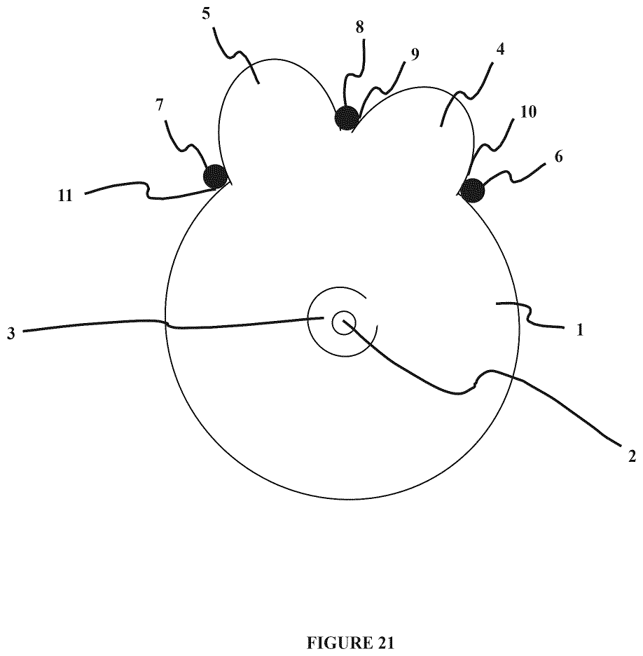

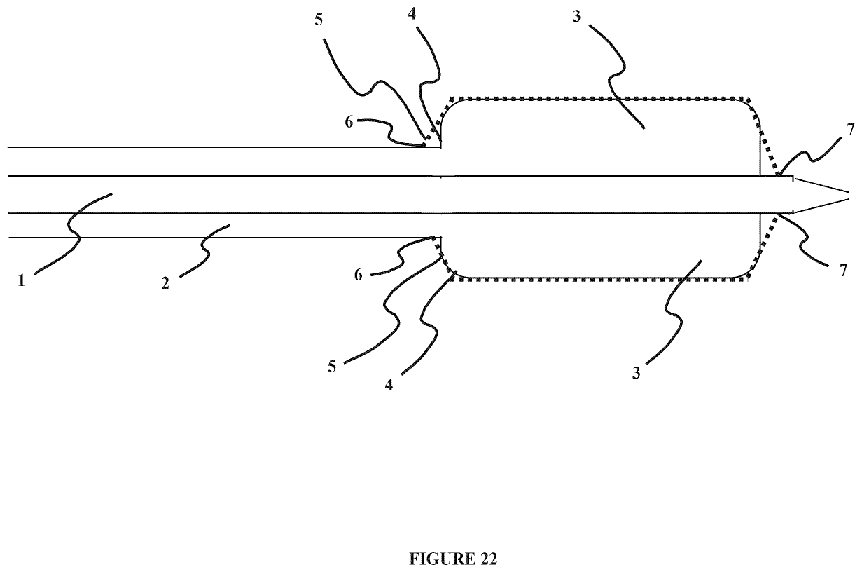

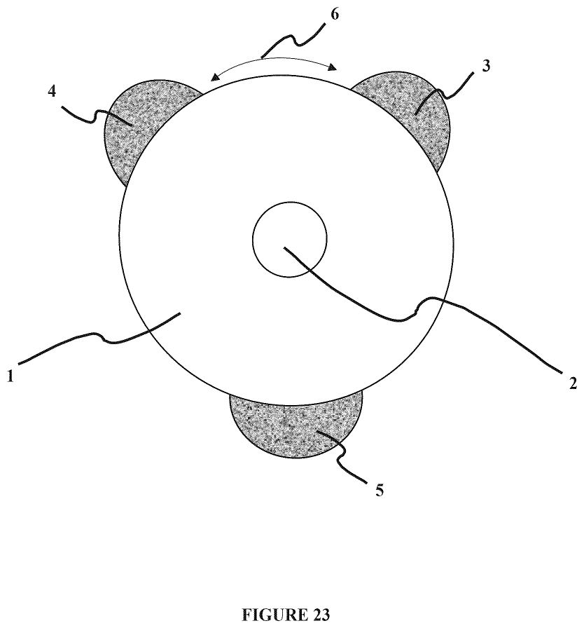

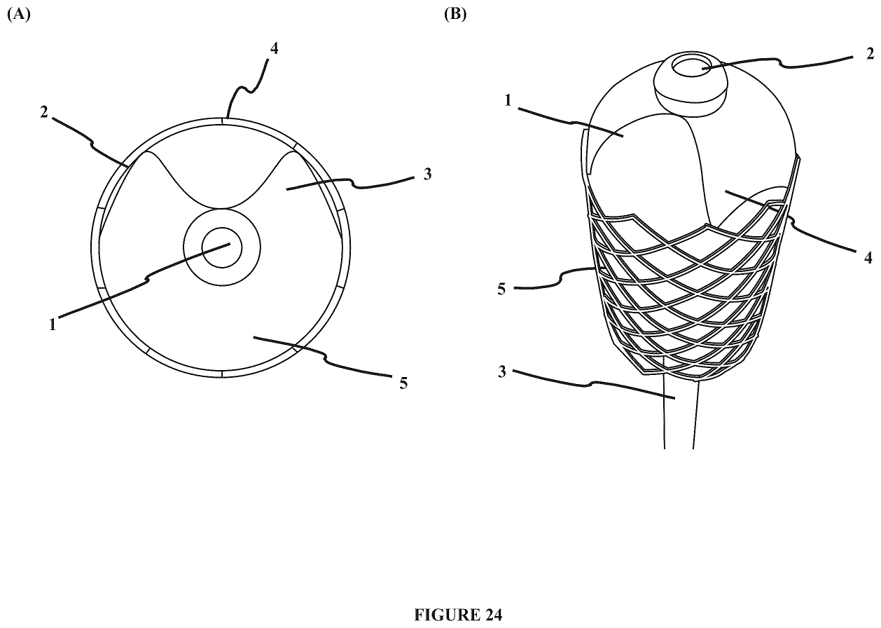

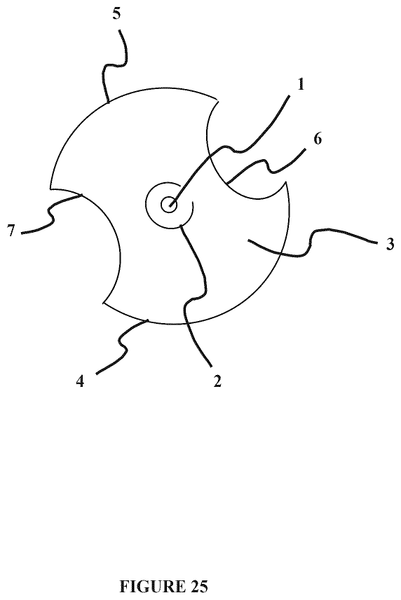

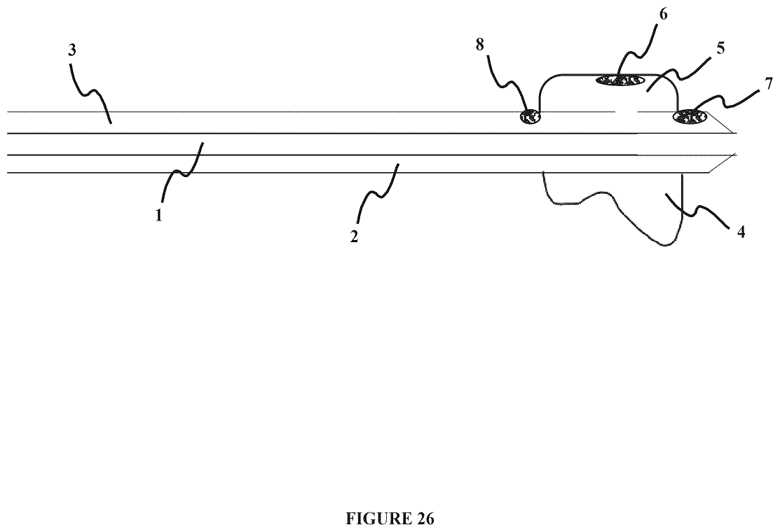

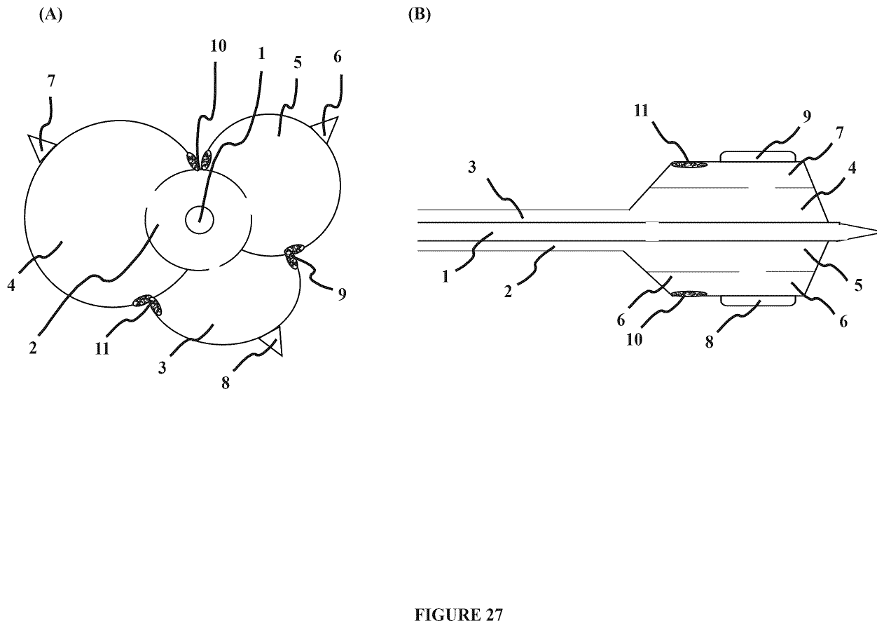

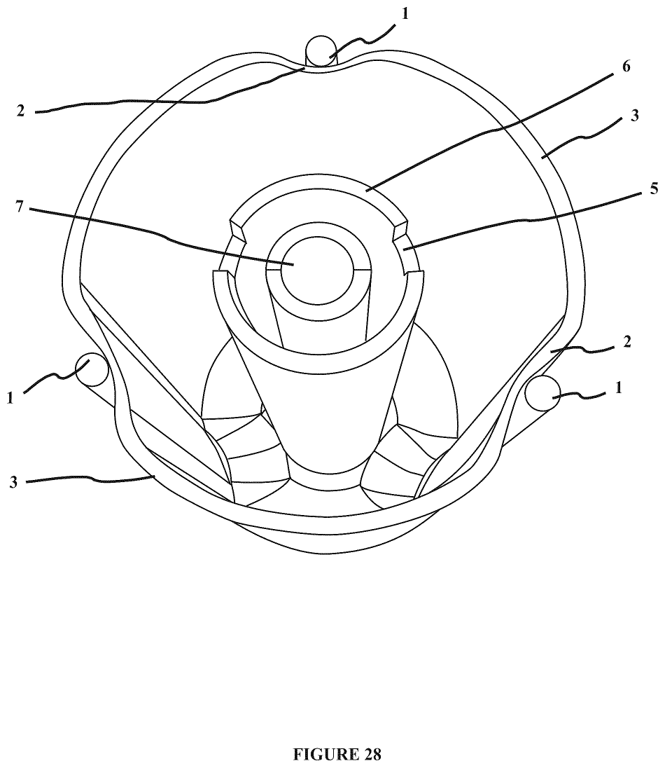

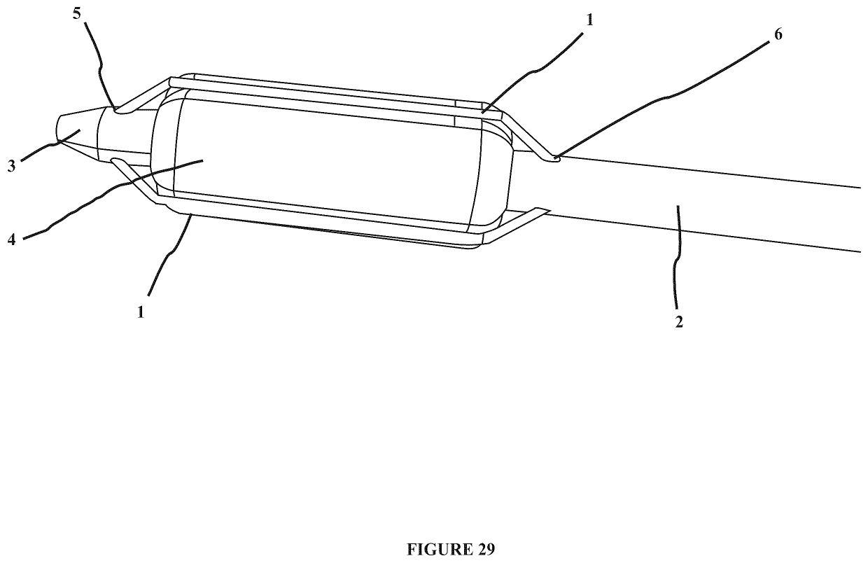

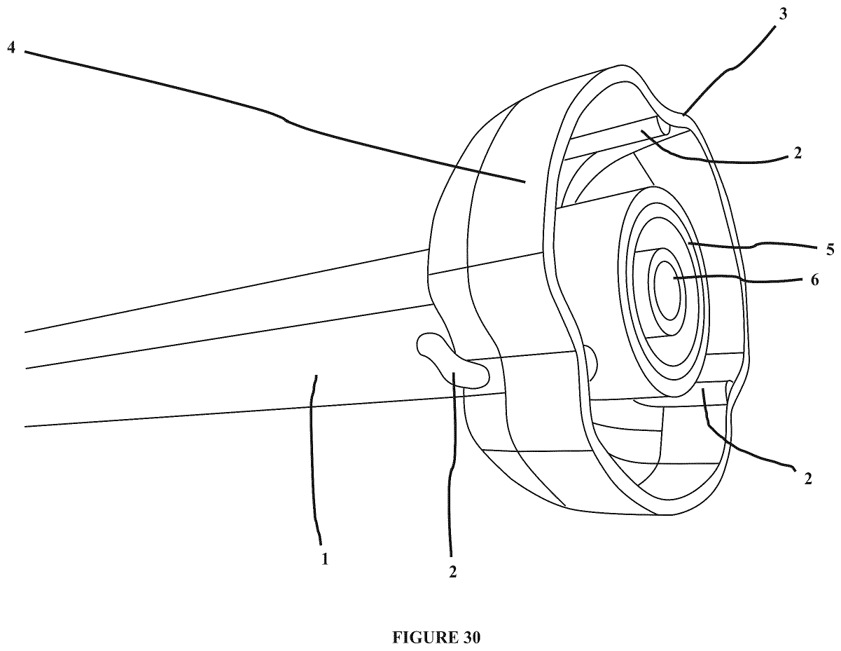

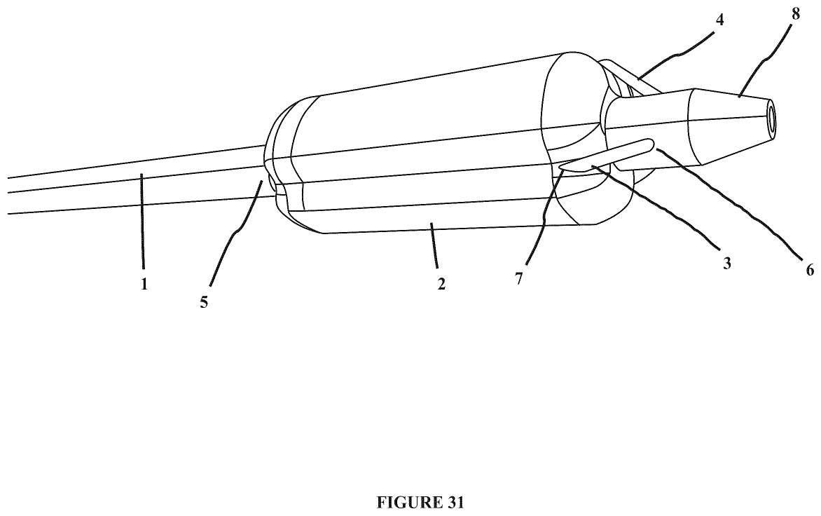

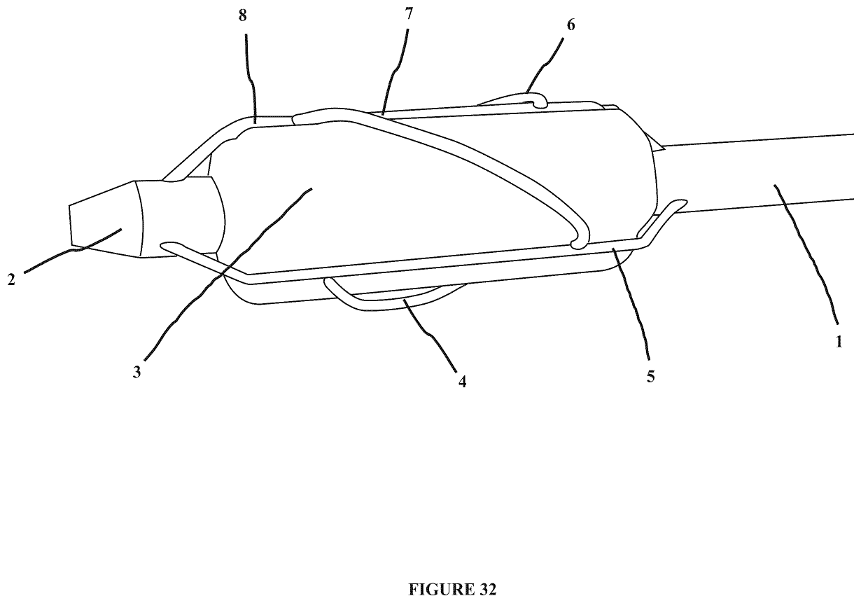

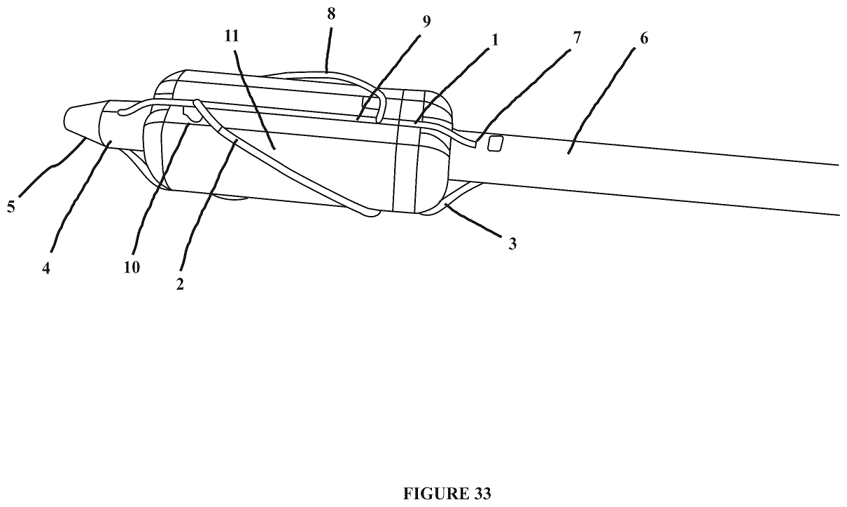

In panel B of FIG. 13, it illustrates one of the embodiments of an inflated device showing a cross-sectional long axis view. In this embodiment, the device has main inflatable chambers (4, 5), with a common inflatable channel (6), that is used to fill all of the main inflatable chambers and also identified is a guidewire channel (2). All of the main inflatable chambers are encased in a larger inflatable chamber (1), which has its own inflation channel (3), the encasing chamber can have various predetermined shapes. In some embodiments, the larger chamber (1) may have no cavity or inflation channel, and may be just made from flexible material that is not fillable. Also identified in the illustration are the Z- and Y-axis of the catheter, the encasing inflation chamber that can be fused in an air-tight bonding to the catheter's shaft and/or the inflation channel, and the encasing chamber may assume a predetermined shape.

FIG. 14 illustrates an embodiment of the present invention and illustrates a representative example of one of the embodiments of an inflated device showing a cross-sectional short axis view. In this embodiment, the device has main inflatable chambers (4) which run parallel to another main inflatable chamber (5) along the Z-axis of the catheter's shaft, both inflatable chambers have their own inflation channels (3, 6), respectively. In this embodiment, there is an overlap (7) between the main inflatable chambers (4, 5) and also identified is a guidewire channel (2).

FIG. 15 illustrates an embodiment of the present invention and illustrates a representative example of one of the embodiments of an inflated device showing a cross-sectional short axis view. In this embodiment, the device has inflatable chamber with protrusions. In this embodiment, inflatable chamber is molded so it has 3 semi-circular protrusions (3, 4, 5). The protrusions' cavities are contiguous with the body of the main inflatable chamber cavity (1), thus only one inflation channel (7) is used to inflate the protrusions as well as the main body of the inflatable chamber. Also identified are the openings (8) between the inflation channel and the main inflation chamber. In this embodiment, the protrusions are distributed evenly in a pattern around the periphery of the main inflatable chamber, and also identified is a guidewire channel (6).

FIG. 16 illustrates an embodiment of the present invention and illustrates a representative example of one of the embodiments of an inflated device showing a cross-sectional long axis view. In this embodiment, the device has a main inflatable chamber (1), with two protrusions (7, 8), with their cavities contiguous with the main inflatable chamber cavity, because the wall between the main inflation chamber (4) and the protrusion has at least one gap (9), while in some embodiments, there is no wall (4) to separate the protrusions from the main inflation chamber. Inflation channel (5) is used for inflation of the main inflation chamber as well as the protrusions, and also identified in the illustration is a guide wire channel (6).

FIG. 17 illustrates an embodiment of the present invention and illustrates a representative example of one of the embodiments of an inflated device showing a cross-sectional short axis view. In this embodiment, the device has inflatable chamber with side inflatable chambers. In this embodiment, main inflatable chamber (12) is surrounded by side chambers (1, 2, 3, 4, 5, 6), side chambers walls can be fused in an air and/or liquid-tight bonding to the main inflatable chamber's wall, or they can have separate walls. Side inflatable chambers can be variable in size, and they can have a pattern around the main catheter's shaft (13, 15). In this embodiment, small side chambers (2, 4, 6) are similar in size and alternate with a similar sized but larger side chambers (1, 3, 5). Each of the side chambers can have their own inflation channels (7, 8, 9, 10, 11, 16). In this embodiment, the main inflation channel (13) is used to inflate main inflation chamber through openings (14) in its wall. Also identified is a guidewire channel (15). Catheter shaft is the combination of the main inflation channel and guidewire channel.

FIG. 18 illustrates an embodiment of the present invention and illustrates a representative example of one of the embodiments of an inflated device showing a cross-sectional longitudinal axis view. In this embodiment, the device has chamber with side inflatable chambers. In this embodiment, main inflatable chamber (1) has two side inflatable chambers (2, 3). Main inflatable chamber has its own inflation channel (7). While the side inflatable chambers (3, 2) have their own inflation channels (4, 5). All inflation channels and the guide wire channel (6) form the catheter's shaft.

FIG. 19 illustrates an embodiment of the present invention and illustrates a representative example of one of the embodiments of an inflated device showing a cross-sectional short axis view. In this embodiment, the device has a main inflatable chamber (1), which has 4 protrusions, two smaller protrusions (4, 5) and 2 larger protrusions (2, 3). Inflation channel (6) is connected to the main inflation chamber. In this embodiment, guide wire channel (6) is inside the main inflation channel. In this embodiment, protrusion (2) has a double layered wall (9, 10). Protrusion (3) has a single layered wall (14). Protrusion (4) has a wall that is partially thicker (13) than the rest of the protrusion wall (12) and thicker than the main inflation chamber wall (8). Protrusion (5) wall (11) is made from different material than other protrusions and different than the main inflation chamber.

FIG. 20 illustrates an embodiment of the present invention and illustrates a representative example of one of the embodiments of an inflated device showing a cross-sectional short axis view. In this embodiment, the device has inflatable chamber with protrusions. X- and Y-axis are illustrated by the dotted lines. In this embodiment, main inflatable chamber (1) has 4 protrusions, two smaller protrusions (4, 5) and 2 larger protrusions (6, 7). Also identified in the illustration are inflation channel (3), a guide wire channel (6) is inside the main inflation channel. Protrusions can have different width (8, 9, 10, 11) at the junction to the main inflation chamber. They also can have different length (14, 15, 16, 17). Maximal width of the protrusions can be smaller than the width at the junction to the main inflatable chamber, or in other embodiment, maximal width is bigger than the width at the junction to the main inflatable chamber (13, 12). In this embodiment, the orientation of the protrusion (5) and protrusion (4) can be variable based on the angle (19, 21) in relation to the Y-axis (or in some embodiment to the X- or Z-axis of the catheter). In some embodiments, the angle (18) and (21) between the protrusions and X-axis (in some other embodiments, Y-axis and the protrusions or between the protrusions themselves) can be variable, while in other embodiments the angles can be the same.

FIG. 21 illustrates an embodiment of the present invention and illustrates a representative example of one of the embodiments of an inflated device showing a cross-sectional short axis view. In this embodiment, the device has inflatable chamber with protrusions and constrictive wires. In this embodiment, expansion of the main inflatable chamber (1) during inflation is restricted in specific locations (9, 10, 11) due to the presence of constrictive wires (7, 8, 6). The constrictive wires in this embodiment are located just outside the inflation chamber. While in other embodiments, the constrictive wires can be embedded inside the inflation chamber's walls, cavity or attached to the outer surface of the inflation chamber's walls. In this embodiment, the presence of constrictive wires resulting in the formation of two protrusions (4, 5) with the expansion of the remainder of the main inflation chamber on the opposite side. The location of the constrictive wires (7, 8, 9) can be pre-defined in relation to the catheter's shaft (guide wire channel (2) and inflation channel (3)). The distribution of the constrictive wires can follow a certain pattern in relation to the catheter's shaft, in this embodiment, one wire is superior (3) with two side wires (6, 7).

FIG. 22 illustrates an embodiment of the present invention and illustrates a representative example of one of the embodiments of an inflated device showing a cross-sectional longitudinal axis view. In this embodiment, the device has inflatable chamber with constrictive wires. In this embodiment, expansion of the main inflatable chamber (3) during inflation is restricted due to the presence of constrictive wires (5). The constrictive wires in this embodiment are located just outside the inflation chamber's walls (4). While in other embodiments, the constrictive wires can be embedded inside the inflation chamber's walls, cavity or attached to the outer surface of the inflation chamber's walls. In this embodiment, the constrictive wires (5) are attached to the catheter's shaft (guide wire channel (1) and inflation channel (2)), the attachment site is typically distal to the inflation chamber (7) and also proximal to the inflation chamber (6).

FIG. 23 illustrates an embodiment of the present invention and illustrates a representative example of one of the embodiments of an inflated device showing a cross-sectional short axis view. In this embodiment, the device has constrictive wires. The present figure illustrates the attachment sites of the constrictive wire, line, tubes etc. to the catheter's shaft. In this embodiment, the catheter' shaft contains inflation channel (1) and also the guidewire channel (2). The constrictive wires (3, 4, 5) are attached to the catheter's shaft at specific locations around the outer surface of the catheter's shaft. The distribution of the attachment sites of the constrictive wires to the catheter's shaft can follow a certain pattern in relation to the catheter's shaft, in this embodiment, the constrictive wires are attached to locations at approximately the same angle (6) to each other when you consider the catheter's shaft as the axis point.

FIG. 24 illustrates an embodiment of the present invention and illustrates in panel A, a representative example of one of the embodiments of an inflated device showing short axis view. In this embodiment, the device has a main inflatable chamber with protrusions, with a stent mounted on them. In this embodiment, main inflatable chamber (5) has 2 protrusions, (2, 3), and a guidewire channel (1). Stent (4) is usually mounted on the inflatable part of the device, which in this embodiment includes the main inflatable chamber, protrusions. Stent is usually compressed against the inflatable chambers, protrusions, side inflatable chambers, when the device is deflated, and then the stent will expand when the device and its components are inflated. In panel B, this figure illustrates a representative example of one of the embodiments of an inflated device showing a long axis view. In this embodiment, the device has inflatable chamber with protrusions, with a stent mounted on them. In this embodiment, (same as panel A of FIG. 24) main inflatable chamber (5) has 2 protrusions (1, 4), and a guidewire channel (2). Stent (5) is usually mounted on the inflatable part of the device, which in this embodiment includes the main inflatable chamber and the protrusions and also shown is a catheter's shaft (3).

FIG. 25 illustrates an embodiment of the present invention and illustrates a representative example of one of the embodiments of an inflated device showing a cross-sectional short axis view. In this embodiment, the device has only one main inflatable chamber (3), and a guidewire channel (1), and an inflation channel (2). The outer surface of the inflatable chamber can assume variety of shapes when inflated. In this embodiment, inflatable chamber's surfaces (5) and (4) are convex, while surfaces (6) and (7) are concave. In other embodiments, these surfaces can be flat, it may have sharp edges etc.

FIG. 26 illustrates an embodiment of the present invention and illustrates a representative example of one of the embodiments of an inflated device showing a cross-sectional long axis view. In this embodiment, the device has inflatable chamber, with radio-opaque markers. In this embodiment, main Inflatable chambers (5) and (4) are attached to the catheter's shaft at the same level, there are radio-opaque markers only one side of the catheter's shaft at the proximal end (8) of the inflatable chamber (5) and also at the distal end of the same inflatable chamber (7), additional radio-opaque marker can embed in the inflatable chamber, such as (6) in this embodiment.

FIG. 27 illustrates an embodiment of the present invention and illustrates in panel A, a representative example of one of the embodiments of an inflated device showing a cross-sectional short axis view. In this embodiment, the device has main inflatable chambers (3, 4, 5). In this embodiment, there are metallic blades attached to each inflatable chamber surface (6, 7, 8). In this embodiment, there are additional metallic components embedded in the walls of the inflatable chambers in specific locations (9, 10, 11). Also identified in the illustration are a guide-wire channel (1), and an inflation channel (2). In panel B this figure illustrates a representative example of one of the embodiments of an inflated device showing a cross-sectional long axis view. In this embodiment, the device has main inflatable chambers (4, 5) with side inflatable chambers (6, 7). In this embodiment, there are metallic blades attached to each inflatable chamber surface (10, 11). In this embodiment, there are additional metallic components embedded in the walls of the inflatable chambers in specific locations (10, 11). Also identified in the illustration are a guide-wire channel (1), and inflation channels (2, 3).

FIG. 28 illustrates an embodiment of the present invention and exemplifies a cross-sectional view of one of the embodiments of the invention, where wires (1) run adjacent to thinner segments of the balloon wall (2), thus creating grooves in the balloon when it is inflated, the remainder of the balloon wall is thicker (3) when compared with the segments that are adjacent to the wires (2). Also identified in the illustration are an inflation channel (6), an inflation hole (5) through which the balloon is inflated, and a wire channel (7).

FIG. 29 illustrates an embodiment of the present invention and exemplifies a cross-sectional view of one of the embodiments of the invention, where wires (1) are attached to the catheter's shaft (2). Also identified in the illustration are a balloon (4), wires attached to the catheter's shaft proximal (6) to the balloon and distal (5) to the balloon, the tip of the catheter (3), where wires create grooves in the balloon by restricting expansion certain parts of the balloon.

FIG. 30 illustrates an embodiment of the present invention and exemplifies a cross-sectional view of one of the embodiments of the invention, where wires (2) are embedded inside the balloon wall (3), thus restricting part of the balloon where they are embedded, and not restricting of other segments of the balloon (3). Also identified in the illustration are wires that are attached to the catheter's shaft (1), an inflation channel (5), and a wire channel (6).

FIG. 31 illustrates an embodiment of the present invention and exemplifies a cross-sectional view of one of the embodiments of the invention, where wires (3, 4) are embedded inside the balloon wall (2), thus restricting part of the balloon where they are embedded, and not restricting the other segments of the balloon. Also identified in the illustration is the tip of the catheter (8), wires that are attached to the catheter's shaft (1) proximally (5) and distally (6), part of the wires may be exposed (3, 4) and are not embedded inside the balloon. In the illustration shown further is a segment of the balloon (7) that is restricted by the embedded wire, while in another segment of the balloon (2) where the wire is not embedded, the balloon can expand more freely.

FIG. 32 illustrates an embodiment of the present invention and exemplifies a cross-sectional view of one of the embodiments of the invention with cross wires, where primary wires (5, 8) are attached to the catheter's shaft, while cross wires (4, 6, 7) connect between the primary wires. Also identified in the illustration is the tip of the catheter (2).

FIG. 33 illustrates an embodiment of the present invention and exemplifies a cross-sectional view of one of the embodiments of the invention with cross wires, where primary wires (1, 3) are attached to the catheter's shaft proximally and distally (7, 4), while cross wires (2, 8) connect between the primary wires. Also identified in the illustration are the tip of the catheter (5), the catheter shaft (6), and the balloon (11). In the illustration shown further is the angle (9, 10) between the cross wire and the primary wire which can range from 0° to 360° degrees.

### DETAILED DESCRIPTION OF THE INVENTION

Detailed embodiments of the present invention are disclosed herein. However, it is to be understood that the disclosed embodiments are merely exemplary of the present invention, which may be embodied in various systems. Therefore, specific details disclosed herein are not to be interpreted as limiting, but rather as a basis for teaching one skilled in the art to variously practice the present invention.

All illustrations of the drawings are to describe selected versions of the present invention and are not intended to limit the scope of the present invention.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

Although any methods and materials similar or equivalent to those described herein can be used in the practice of the present invention, the exemplary methods, devices, and materials are described herein. For the present disclosure, the following terms are defined below. Additional definitions are set forth throughout this disclosure.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a microbe, a microbial formulation, a pharmaceutical composition, and/or a method that “comprises” a list of elements (e.g., components, features, or steps) is not necessarily limited to only those elements (or components or steps), but may include other elements (or components or steps) not expressly listed or inherent to the microbe, microbial formulation, pharmaceutical composition and/or method. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “and/or” when used in a list of two or more items, means that any one of the listed items can be employed by itself or in combination with any one or more of the listed items. For example, the expression “A and/or B” is intended to mean either or both of A and B, i.e., A alone, B alone or A and B in combination. The expression “A, B and/or C” is intended to mean A alone, B alone, C alone, A and B in combination, A and C in combination, B and C in combination or A, B, and C in combination.

As used herein, the term “about” refers to a rough estimate of the number or amount of the quantity referred to and is in the vicinity of the actual number or figure immediately following said term, where the actual number or figure or amount could be slightly higher or lower.

As used herein, the term “protrusion” refers to a bulging of an inflation chamber periphery, with the wall of the protrusion that is contiguous with the inflatable chamber's wall, typically a main inflatable chamber. For instance, FIG. 15 shows protrusions that can be filled at the same time as the inflatable chamber, which they are associated with. Inflation is done using the shared inflation channel.

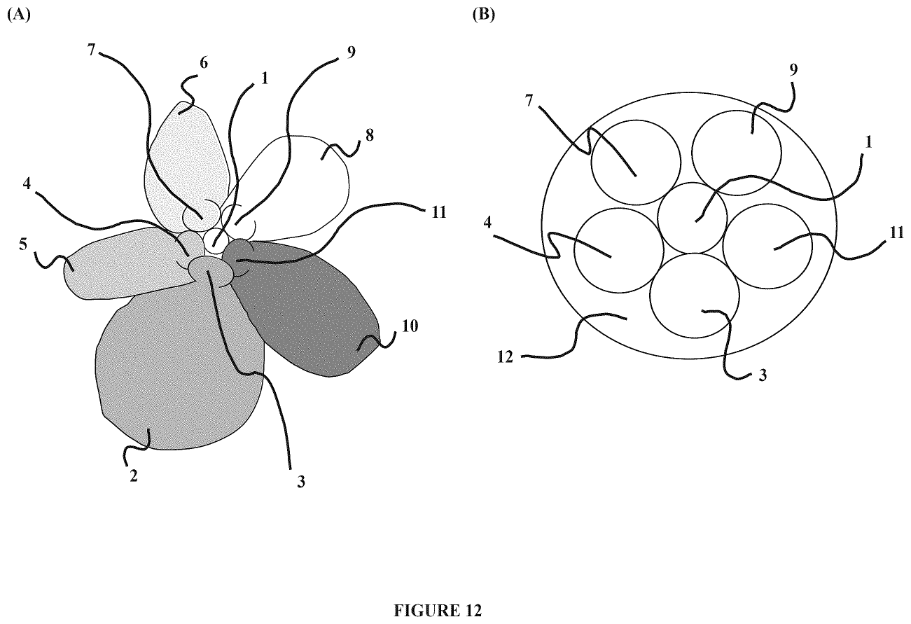

As used herein, the term a “side inflation chamber” refers to a chamber that has a wall that partially or completely separates it from the main inflatable chamber, and it can have its own inflatable channel, or it may share an inflatable channel with a main inflatable chamber as illustrated in FIG. 12 in panel A. A main inflatable chamber is a chamber that is attached to the catheter's shaft or shares a wall with the catheter's shaft as illustrated in FIG. 11 in panel A.

As used herein, the term “main inflatable chamber” refers to a chamber that is attached to the catheter's shaft or shares a wall with the catheter's shaft as illustrated in FIG. 11 in panel A.

As discussed above, there remains a need in the art for better inflatable devices such as coronary and peripheral angioplasty devices, which can achieve better expansion of blockages in various types of blood vessels, and expansion of other cardiac and non-chambers and/or channels in the body and one that can be used in other inflatable devices that can be used to open other blockages in cardiology, urology, gastrointestinal, biliary ducts, non-medical blockages etc.

The present invention addresses the aforementioned need and provides a shaped inflatable apparatus, system, and/or device with a new design of the inflatable chamber part for better expansion of blockages in various types of blood vessels, and expansion of other cardiac and non-cardiac chambers and/or channels in the body with the use of non-cylindrical (non-circular in cross-section of the device) shape to enable the more precise application of the force and energy needed to expand a narrowing in the blood vessel, aperture, chamber, etc. to open blockages in cardiology, urology, gastrointestinal, biliary ducts, non-medical blockages etc.

In one of the embodiments of the present invention, it provides a shaped inflatable apparatus, system, and/or device for treating pathological narrowing of and/or expansion of blockages in fluid-carrying conduits such as blood vessels in the body of an animal, the inflatable apparatus, system, and/or device comprises an inflatable chamber, wherein the inflatable chamber provides for better expansion of blockages in various types of blood vessels, and expansion of other cardiac and non-chambers and/or channels in the body, wherein the inflatable chamber is the non-cylindrical shape or non-circular in cross-section of the apparatus, system, and/or device, wherein the animal is a mammal, and wherein the mammal is a human.

In another embodiment of the present invention, it provides a shaped inflatable apparatus, system, and/or device as disclosed hereinabove, wherein the inflatable chamber achieves better distribution of the force being applied and allows the use of a lower nominal pressure of the inflatable device to reduce acute complications.

In an embodiment of the present invention, it provides a shaped inflatable apparatus, system, and/or device for treating pathological narrowing of and/or expansion of blockages in fluid-carrying conduits such as blood vessels or in other non-fluid carrying conduits in a body of an animal, the inflatable apparatus, system, and/or device comprises a shaft; an inflation channel(s); one or more guide-wire channel(s); one or more stiffening wire; an inflation port(s); one or more guide-wire port; and a shaft tip; and optionally it further comprises a varying combination of parts selected from a group comprising one or more protrusions, wherein the protrusions are bulging of an inflation chamber periphery, with the wall of the protrusion that is contiguous with the inflatable chamber's wall, typically a main inflatable chamber to allow same time inflation; one or more side inflation chambers, wherein the side inflatable chambers are the ones with a wall that partially or completely separates them from the main inflatable chamber, and optionally they have their own inflatable channel, and/or they share an inflatable channel with a main inflatable chamber; one or more shafts made from flexible material, and is typically non-inflatable.

In another embodiment of the present invention, it provides a shaped inflatable apparatus, system, and/or device as disclosed hereinabove, wherein the inflatable chamber comprises of typically known material for the inflatable chamber's walls that are typically made from flexible inflatable material such as polyvinyl chloride (PVC), polyethylene (PE), polyethylene terephthalate (PET), polyolefin copolymers (POC), nylon derivatives (N), nylon-rein-forced polyurethane (PU), co-extruded copolymers (CP), etc. Other materials such as metallic elements, alloys, etc. can be infused, attached, fused, or impregnated in the walls of the inflatable chamber for added functionality.

The present invention as disclosed herein may also have one or more of the following components:

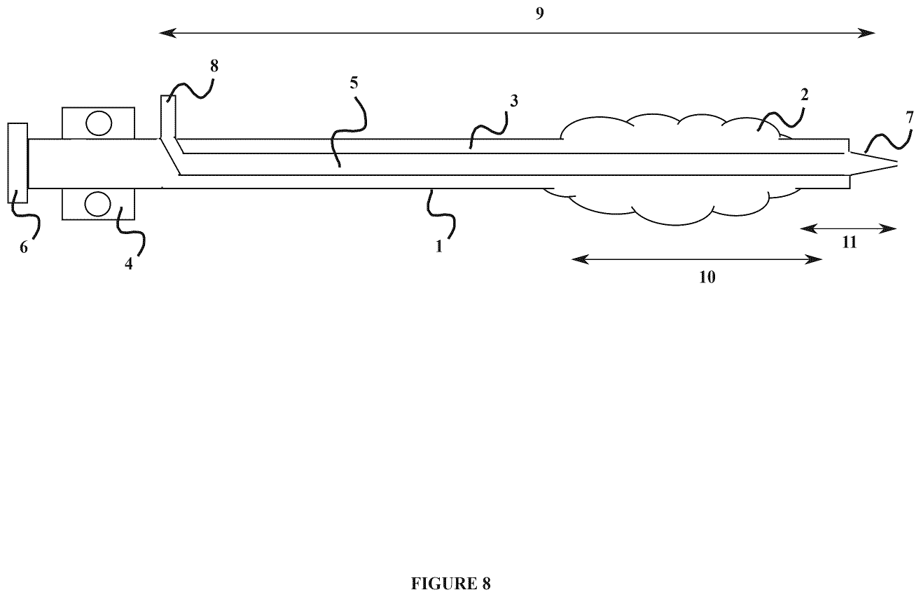

The shaft which is typically made from a flexible material, contains the major components of the device and is used as the main structure of the device. It is typically inserted into the patient using a special sheath into the peripheral vessel. The shaft is typically cylindrical in shape and itself is typically non-inflatable as illustrated in FIG. 8. The shaft typically has one or more ports that are connected to the different channels inside the shaft. These ports can be connected to inflation devices, syringes, manometers (to measure pressure), etc.

One or more inflatable channels are located inside the device's shaft and run along the entire length or part of the device's shaft, and they are used for inflating the inflatable chamber(s). They are typically air-tight and fluid-tight to limit leaks. They are connected to inflation port(s) on one side and the inflation chamber(s) on the other side as illustrated in FIG. 8.

One or more guide wire(s) channel(s) are located inside the device's shaft and run along the entire or part of the length of the device, and used for passing guide wire(s) inside the device typically to the tip of the device or through other openings anywhere along the length of the device's shaft as illustrated in FIG. 8. Guidewire channels are connected to guide-wire port(s) that are attached to the device's shaft, which is typically outside the patient when the device is inserted inside the body.

Inflatable chamber(s) or Inflation chamber(s) used interchangeably, is attached, typically with its walls partially fused, to the device's shaft (typically to the outside of the device's shaft), and connected to the inflating channel(s). The attachment to the catheter's shaft is typically gas and or liquid tight to void leaks as illustrated in FIG. 8. Inflatable chamber's walls are typically made from flexible inflatable material such as polyvinyl chloride (PVC), polyethylene (PE), polyethylene terephthalate (PET), polyolefin copolymers (POC), nylon derivatives (N), nylon-rein-forced polyurethane (PU), co-extruded copolymers (CP), etc. The wall of the inflatable chamber can be formed from one or more layers of the same or different material(s) to enable the shaping of the device. Other materials such as metallic elements, alloys, etc. can be infused or impregnated in the walls of the inflatable chamber for added functionality. Inflatable chamber(s) are usually folded around the shaft of the device when they are deflated to minimize the overall diameter of the device, and also to enable insertion in smaller channels such as peripheral blood vessels, etc.

The diameter of the shaft and attached inflation chamber(s) is typically very small (<5 cm) so it can be inserted into small opening in the blood vessel or other channels through the body.

The device is usually advanced inside a blood vessel, body channel or orifice into the target area using the guide wire, under fluoroscopy guidance. Then once the device is verified to be at the correct position, then the inflatable chamber(s) is inflated using gas and/or liquid to a certain pressure and/or a certain volume to achieve dilatation of the target organ while minimizing the risk of rupture of the inflatable chamber. Inflation is usually done using a special inflation device, syringe, which may have one or pressure measuring sensors and/or manometer. One or more inflation devices can be used in the process, each can be connected to specific inflation channel inside the shaft which are connected to specific inflatable chamber.

The present invention discloses shaped inflatable apparatus, system, and/or device as disclosed hereinabove, where the shaped inflatable apparatus, system, and/or device has a different shape other than cylindrical shape that is circular in cross section when inflated. When the inflatable chamber is inflated, then its shape can be oblong, oval, star-like with rounded edges, triangular with rounded edge etc. as viewed from the X, Y, and/or Z axis of the device's shaft. The inflation chamber can be symmetric or asymmetric in relation to the device's shaft (and/or guidewire channel), along the X or Y or Z axis of the shaft, so that the device's shaft and/or the guidewire channel is not in the center of the inflatable chamber(s). Length and depth of inflatable chamber can be variable. Also, the guidewire channel can be to the side of the inflatable channel (adjacent to the inflatable chamber's wall) with zero width between the wall of the inflatable channel and the guidewire channel, or it can be attached to the outside of the inflatable channel walls. And the non-cylindrical shape of the inflatable chamber(s) can be present when the device inflated, or deflated or both. Further the inflatable devices or balloons can be angulated (when inflated and/or deflated) when compared to the device's shaft and/or guidewire channel, the angle can be anywhere from 0° to 359° degrees.

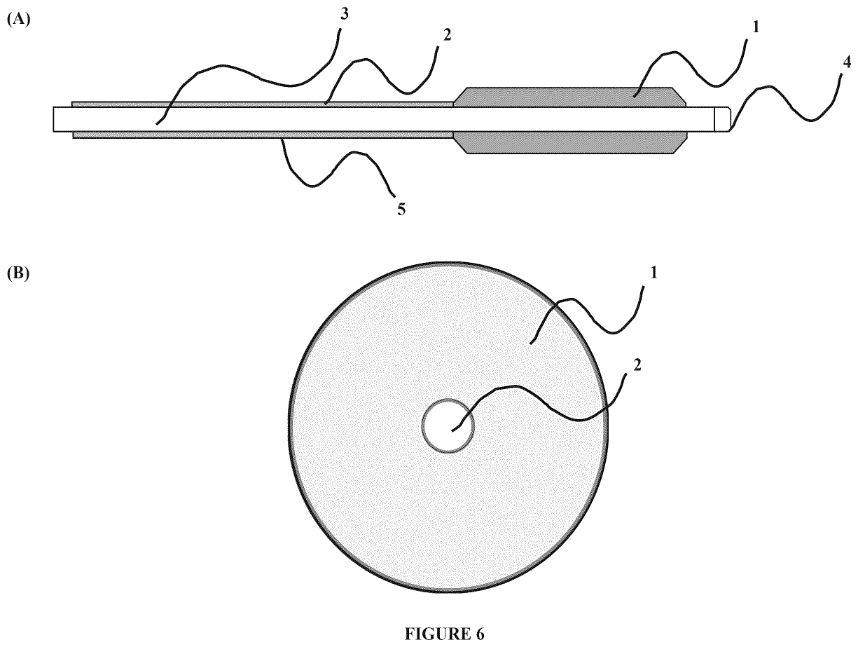

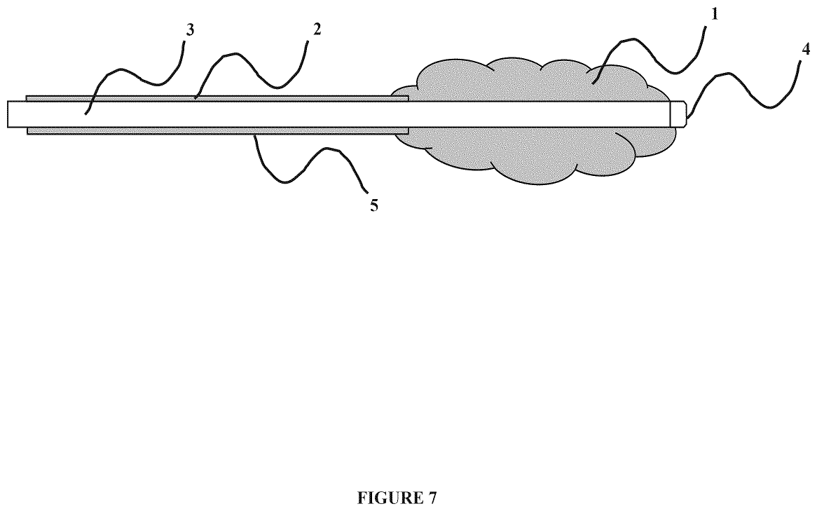

In some embodiments of the present invention as disclosed herein, the inflatable device such as balloon, may assume a non-cylindrical (non-circular in cross section of the device) shape to enable more precise application of the force and energy needed to expand a narrowing in the blood vessel, aperture, chamber, etc. The present invention as disclosed herein is used to replace the balloon (inflatable part) of current and future angioplasty devices regardless of their type, such as rapid exchange balloon catheter, over-the-wire balloon catheter, fixed guidewire balloon catheter, perfusion balloon catheters etc. as have been illustrated by a representative example in FIG. 6 in its panel A and B.

For ease of description, in the present invention as disclosed herein, inflatable chamber is used interchangeably with a balloon and also catheter is used interchangeably with a device. The inflatable chamber(s) is connected through a gas and/or fluid tight channel called an inflation channel, which is located inside the delivery catheter's shaft, to enable filling of the balloon externally using a plunger or other inflating device or inflating mechanism. Usually, another channel, guidewire channel, is present inside the device's shaft and extend out till the tip of the device, and it is used to advance a guidewire, which is used to advance the catheter into the correct position.

The catheter (device) is inserted typically through a small hole in the blood vessel. Once the correct position is achieved, then progressive amount of gas and/or liquid is injected using an inflation device that is attached to the inflation channel, through an inflation port, to inflate the balloon. Expansion of the balloon is typically done under fluoroscopy guidance, until a certain pressure is achieved and/or maximal volume is injected inside the balloon through the delivery catheter and/or until the narrowing in the target vessel is expanded to a certain degree. Typically, ambient air is used for inflation, but in some other embodiments, other types of gases and/or liquids and/or combination of gas and liquid can be used instead for inflation. Inflation process is typically stopped once the target vessel's narrowed segment or aperture or chamber is expanded to the optimal size, or a certain maximal inflation volume and/or inflation pressure is achieved.

The goal of the present invention as disclosed herein is to achieve optimal dilatation of the target vessel, body channel, aperture or chamber, typically by using a lower nominal pressure of the inflatable device to reduce acute complications.

The present invention as disclosed herein can be used in the treatment of coronary artery disease, peripheral vascular disease, carotid artery stenosis and other vascular diseases. It also can be used in urology, gastrointestinal disease, hepato-biliary diseases and other medical illnesses, in addition to that it can be used in non-medical applications.

The embodiments of the present invention as disclosed herein can be used, individually or in any combination(s), as a part of any interventional device or used as a modification to the device. The devices of the present invention can be any interventional medical device or other occluder or functional device used in other industry such as oil industry.

A typical standard balloon angioplasty device in use conventionally consists of a balloon or inflatable chamber mounted on a catheter shaft. The shaft typically contains an inflating channel surrounding a central guidewire channel, which is used to advance a guide wire. At the end of the catheter shaft, there is an inflation port which is connected to the inflating channel, and it is used for inflation of the balloon. Walls of the inflatable chamber are flexible, and expand with additional inflation volume and/or pressure. While the walls of the main shaft of the catheter are flexible but they usually do not expand during the inflation process as shown in the representative illustration in FIG. 6 in panel A.

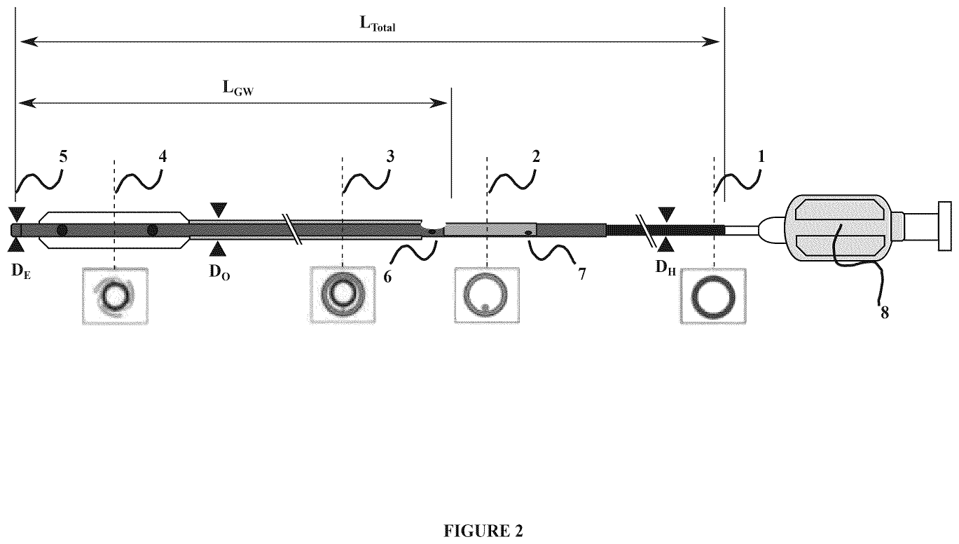

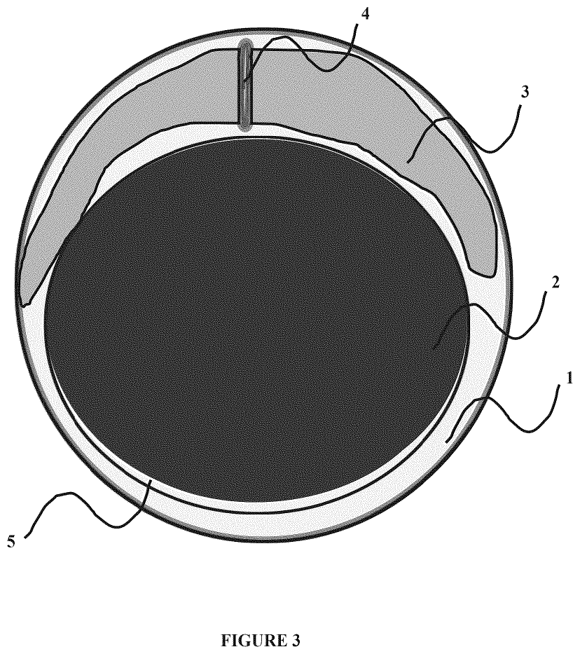

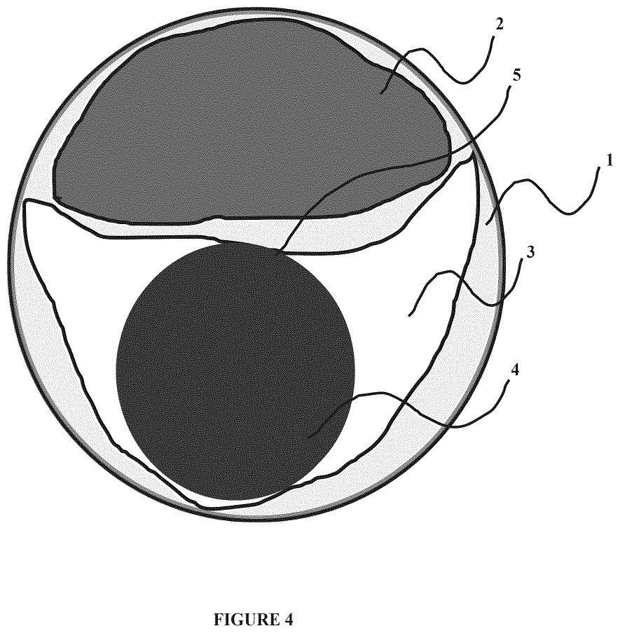

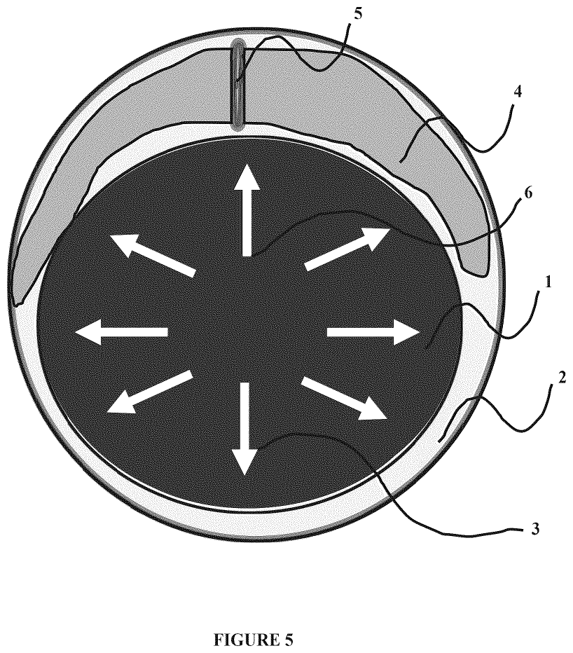

The present invention as disclosed herein describes a new design of the inflatable chamber part of the angioplasty devices and/or other inflatable medical devices, where the balloon may assume a different shape other than standard cylindrical shape (circular in cross-section), refer to FIG. 6 in panel B, when inflated, to achieve better distribution of the force being applied to the vessel wall and/or the target aperture, channel, or chamber. In some embodiments, the present invention as disclosed herein uses the basic design of current angioplasty devices, as described above, where it may contain any one or more of the following components: Shaft, inflation channel(s), one or more guide-wire channel(s), stiffening wire, inflation port(s), guide-wire port, shaft tip etc. as illustrated in FIG. 7, and in FIG. 2.

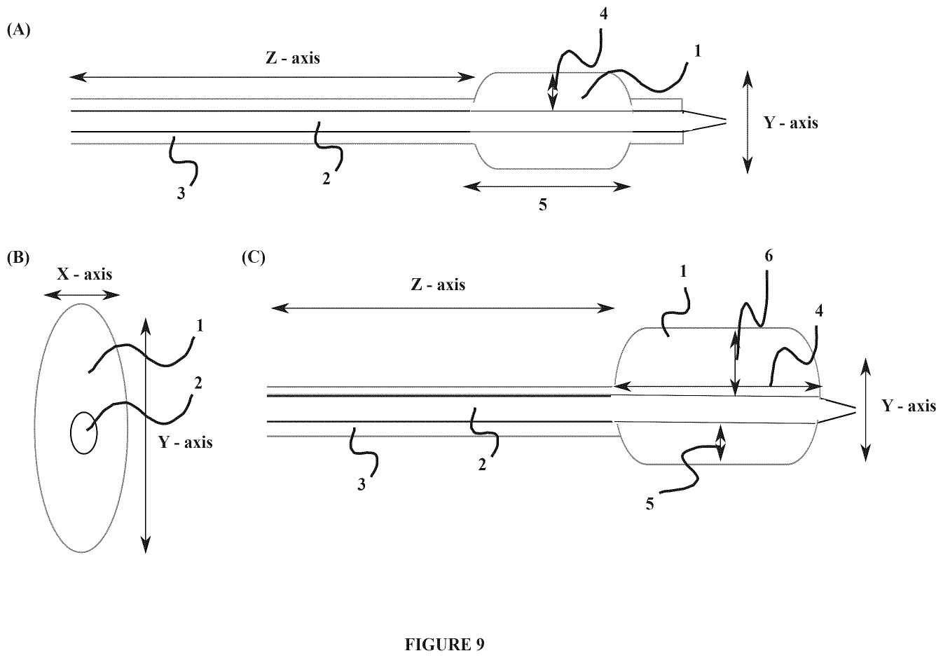

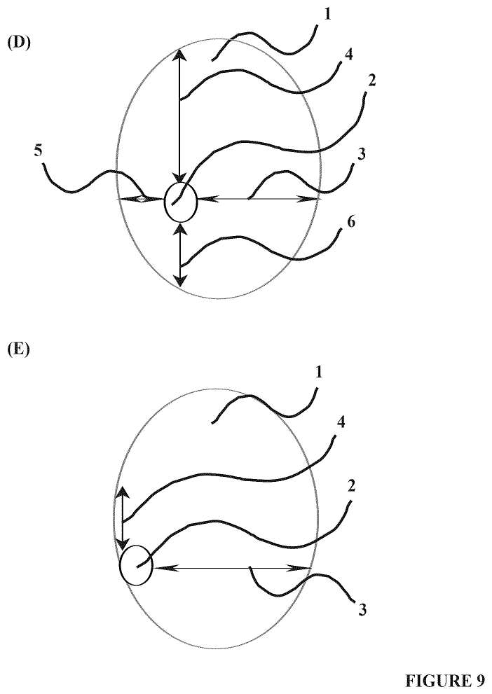

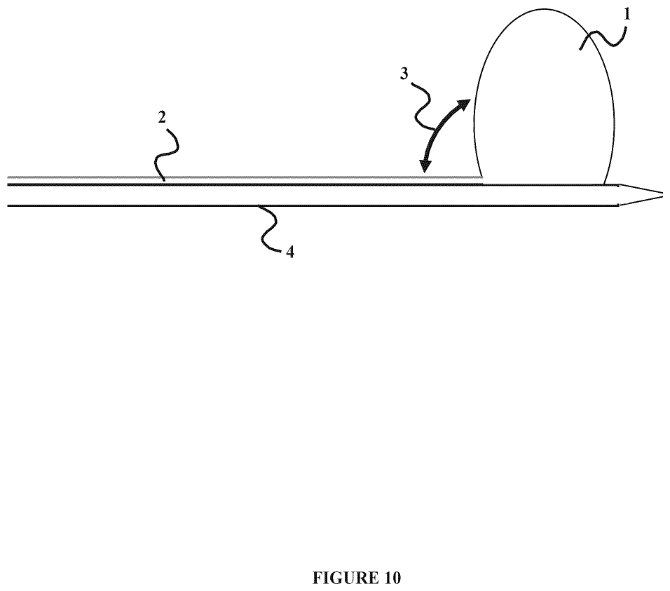

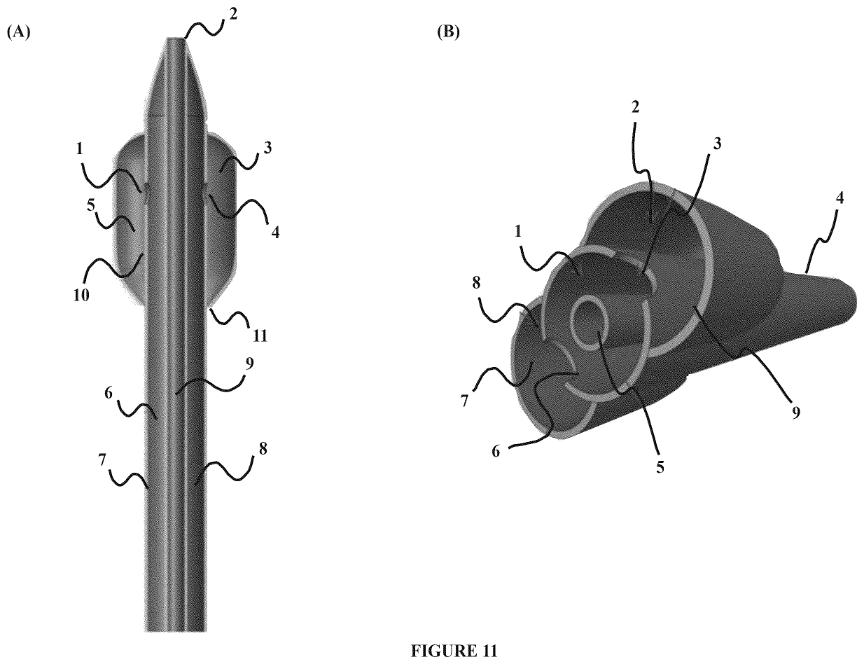

In an embodiment of the present invention, the main inflatable chamber (or balloon) of the angioplasty device may assume a different shape other than cylindrical shape (circular in cross section) when inflated. When the inflatable chamber is inflated, then its shape can be oblong, oval, as illustrated in FIG. 9A in panel B, star-like with rounded edges, as illustrated in FIG. 15, triangular with rounded edge etc. as viewed from the X, Y, and/or Z axis of the device's shaft. The inflation chamber can be symmetric or asymmetric in relation to the device's shaft (and/or guidewire channel), along the X or Y or Z axis of the shaft, so that the device's shaft and/or the guidewire channel is not in the center of the inflatable chamber(s). Length and depth of inflatable chamber can be variable, refer to FIG. 9A in panels A to C and FIG. 9B in panel D. In some embodiments, the guidewire channel can be to the side of the inflatable channel (adjacent to the inflatable chamber's wall) with zero width between the wall of the inflatable channel and the guidewire channel, or it can be attached to the outside of the inflatable channel walls as illustrated in FIG. 9B in panel E.

In some embodiments of the present invention as disclosed herein, the non-cylindrical shape of the inflatable chamber(s) can be present when the device inflated, or deflated or both.

In some embodiments of the present invention as disclosed herein, the balloons can be angulated (when inflated and/or deflated) when compared to the device's shaft and/or guidewire channel, the angle can be anywhere from 0° to 359° degrees as illustrated in FIG. 10.

In some embodiments of the present invention as disclosed herein, shaped inflatable balloon can be made by molding the inflatable chamber, so it will have a non-cylindrical shape when inflated and/or deflated.

In some embodiments of the present invention as disclosed herein, Inflatable chambers can come in different sizes to match the chamber, vessel, channel, aperture that needs to be dilated.

In some embodiments of the present invention as disclosed herein, various devices can be made in various sizes, with various minimal and maximal widths and/or maximal and minimal length of each inflatable chamber, as measured if the device is inflated and/or deflated as illustrated in FIG. 9A in panel A to C and FIG. 9B in panel D. Various sizes may be necessary to dilate various sizes of chambers, channels, apertures etc.

In some embodiments of the present invention as disclosed herein inflatable chambers are typically fused or attached with gas-tight and/or liquid-tight bonding to the inflation channel(s) to prevent any leaks.

In some embodiments of the present invention as disclosed herein, two or more main inflatable chambers can be attached to the catheter's shaft. They are attached or fused with gas-tight and/or liquid-tight bonding to the inflation channels and/or catheter's shaft to prevent any leaks. Main inflatable chambers are typically filled through opening(s) in inflatable channel(s). All of the main inflatable chambers can be connected to one or more common inflation channels, which extends into the catheter's shaft as illustrated in FIG. 11 in panels A, and B. In other embodiments, one or more main inflatable chambers can be connected to one or more separate inflation channel, which extend into the catheter's shaft as illustrated in FIG. 12 in panel A.

In some embodiments of the present invention as disclosed herein, the main inflatable channels can be similar in shape and size, or they may have different shapes and size, which can follow a certain pattern in relation to the catheter's shaft. For example, refer to FIG. 12 in panel B for example where a large balloon followed by a smaller balloon followed by a large balloon around the catheter's shaft.

In some embodiments of the present invention as disclosed herein, one or more main inflatable chambers can be encased in a larger inflatable chamber that can be filled through its own infatuation channel, and/or a common inflation channel. As illustrated in FIG. 13 in panels A, and B, where the encasing inflation chamber is fused in an air-tight bonding to the catheter' shaft and/or the inflation channel and/or guidewire channel. In other embodiment, one or more main inflatable chambers can be encased in a larger non-inflatable chamber made from flexible material such as polyvinyl chloride (PVC), polyethylene (PE), polyethylene terephthalate (PET), polyolefin copolymers (POC), nylon derivatives (N), nylon-rein-forced polyurethane (PU), co-extruded copolymers (CP), etc. Encasing chamber may have different shapes based on the functionality required.

In some embodiments of the present invention as disclosed herein, main Inflatable chambers can be attached to one or more shared inflation channel(s), which extend into the device's shaft, and connected to one or more outside inflation hubs as illustrated in FIG. 11 in panel B. One or more Inflatable chamber(s) can have their own inflation channel(s) that extend into the driver's shaft, and are connected to one or more outside inflation hubs as illustrated in FIG. 12 in panels A and B.

In some embodiments of the present invention as disclosed herein, there can be one or more inflatable main chamber(s), Main inflatable chamber(s) can run parallel to each other along the device's shaft, or they can run serially along the length of the device's shaft. There can be no, partial or complete overlap between the inflatable chambers along the Z-axis, X axis and/or Y axis of the device, refer to FIG. 14.

In some embodiments of the present invention as disclosed herein, there can be one or more side inflatable chambers and/or one or more protrusion(s) out of the periphery of the main inflatable chambers. Side chambers and or protrusions can be present when the device is being inflated and/or deflated.

In some embodiments of the present invention as disclosed herein, protrusions can be formed by making a molded balloon with shaped profile that have protrusions and/or side inflatable chambers, when inflated and/or deflated. Refer to FIGS. 15, 16, 17, and 18 as disclosed.

In some embodiments of the present invention as disclosed herein, any one or more protrusions can have walls with different physical properties (in some part(s) of the protrusion, or the entire protrusion) when compared with the main inflatable chamber that they are associated with (such as thickness, density of the protrusions' wall, number of layers that form the protrusion wall etc.). Protrusions' wall can have different number of layers that form the wall, and/or their composition when compared with other part of the inflatable chamber. In some embodiments, a protrusion is formed due to using a thinner wall at the junction between the protrusion and the inflatable chamber, or using material that is more compliant. In other embodiments, a protrusion can have overall or partially thinner wall and/or built using more compliant material, so it can expand faster than the main inflatable chamber, with the opposite can be done to build the protrusion with thicker material or less compliant, or using other variation in physical or mechanical properties of the walls of the protrusions or the main inflation chamber(s) as illustrated in FIG. 19.

In some embodiments of the present invention as disclosed herein, any one or more of the protrusions can have walls with different mechanical properties (in some part(s) of the protrusion, or the entire protrusion) when compared with the main inflatable chamber (such as elasticity, strength, etc.). This can be due to the use of one or more different material(s) with different mechanical properties, to build the protrusion wall, than what is used to build other segments of the main inflatable chambers, or it can be due to the use of different material composition of the protrusions' wall when compared with the remainder of the inflatable chamber (refer to FIG. 19), or due to the addition of other materials such as metallic elements or alloys etc. that can be embedded or attached to the protrusions' walls to change the mechanical properties of the protrusions' wall.

In some embodiments of the present invention as disclosed herein, protrusions may have similar mechanical, physical, and/or material properties among themselves, or they may have different properties among themselves, which can be different than the mechanical properties of the main inflation chamber as illustrated in FIG. 19.

In some embodiments of the present invention as disclosed herein, side chambers' walls can have the same variation in physical and/or mechanical properties or materials used to build them in similar fashion to what is described above for the protrusions.

In some embodiments of the present invention as disclosed herein, any one or more protrusion and/or side chamber can be round, oval, finger-like, irregular or any other shape. In the same device, protrusions and side chambers can be similar in size and shape, but they can be variable size and shape. Refer to FIG. 12 in panel A, FIG. 13 in panel A, FIGS. 15, 17, and 19. This variability in shape and/or size can follow a certain pattern in relationship to the catheter's X-, Y- and Z-axis.

In some embodiments of the present invention as disclosed herein, each protrusion and side chamber can be varied in maximal and minimal width, length, location, orientation in relation to the device shaft's X, Y and Z axis and to other inflatable chamber as illustrated in FIG. 20.

In some embodiments of the present invention as disclosed herein, the protrusions (when inflated) as disclosed herein can apply a higher pressure per surface area and/or higher sheer stress to the diseased segments of the vessel wall or chamber wall, when compared with other parts of the inflatable device when inflated, at the same time, minimizing the pressure being applied to the normal part of the target vessel or chamber wall, thus it can improve the distribution of force being applied and better dilate the vessel or chamber. The location (in relation to the inflatable chamber) and/or the size of the protrusion(s) and/or the shape may be distributed in a pattern along the perimeter and/or length of the inflatable chamber. Refer to FIGS. 12 in panel A, FIG. 13 in panel A, FIGS. 15, and 17.

In some embodiments of the present invention as disclosed herein, the protrusions and/or side chambers may be similar in shape and/or size, or vary in size and/or shape when inflated according to the pressure or the volume of gas and/or liquid used to inflate them.

In some embodiments of the present invention as disclosed herein, any protrusion(s) and/or side chamber(s) can expand at the same speed as the main body of the inflatable chamber, or it may be more resistant to inflation due to the different physical or mechanical properties of the walls of the protrusions and/or side chambers or any segments within them, just as described earlier. for example, using more compliant material to build the protrusion, while using non-compliant material to build the main inflatable chamber can result in different expansion rate based on the volume and or pressure of gas and or liquid being used for inflation.

In some embodiments of the present invention as disclosed herein, the device may have constrictive wires, lines, cylinders, tubes etc. made from one or more of the following: non-flexible, semi-flexible material, memory metal such as Nitinol, other metals such as stainless steel or titanium etc. One or more constrictive wires, lines, cylinders, or tubes can be used to build the device. All of the constrictive wires, lines, cylinders, or tubes can be made from the same material, or one or more can be built using different materials. One or more constrictive wires, lines, cylinders, or tubes can be embedded, fused or attached to the inflatable chamber walls, protrusions' walls, and/or side chamber walls. While in other embodiments, the constrictive wires, lines, cylinders, or tubes can be located on the outside of the inflatable chamber walls, protrusions' walls, side chamber walls. In some embodiments, when the device is inflated, then the constrictive wires, lines, cylinders, or tubes etc. will result in restriction of the expansion of inflatable chamber in certain location, thus can help in creating a shaped inflatable device. The constrictive wires, lines, cylinders, or tubes are atypically attached to the catheter's shaft on both side of the inflatable chambers. Refer to FIGS. 21, 22, and 23. The constrictive wires, lines, cylinders, or tubes can be attached to specific location(s) of the catheter's shaft to give the device specific shape, the distribution of the wires around the inflation catheter can follow a certain pattern around and in relation to the catheter's shaft. Refer to FIGS. 21, 22, and 23.

In some embodiments of the present invention as disclosed herein, the guide wire channel can run in the center of the shaft all the way to the tip of the device, with the Inflation channel(s) surrounding the guide wire channel. In other embodiments, the guide wire channel(s) may parallel and/or adjacent to the inflation channel(s) inside the catheter's shaft.

In some embodiments of the present invention as disclosed herein, the device may consist of one or more main inflatable chambers, which can be attached to the device's shaft, with additional one or more side inflatable chambers that are attached to, fused to, or located adjacent to the outer part of the main inflatable chambers, or built into the main inflatable chamber. These additional side chambers may have their own inflation channels that run through the shaft of the device as illustrated in FIG. 18. In some embodiments, one or more side inflatable chambers can be attached to a shared inflation channel, which can be shared with the inflation channel that is connected to the main inflatable chamber. Any one or more of these inflation channels can be connected to its own separate inflation port, or in some embodiments two or more inflation channels can have a shared inflation port.

In some embodiments of the present invention as disclosed herein, the shape of the inflatable chamber(s), protrusions, side inflatable chambers may change during the inflation process dependent on the volume and/or pressure of gas and/or fluid that is used for inflation.

In some embodiments of the present invention as disclosed herein, the device may have one or more main inflatable chamber(s) which assumes a non-cylindrical shape when inflated. The irregular shape of the inflatable chamber may assume a patterned shape or non-patterned shape. In some embodiments, the inflatable chamber may have a round side with additional protrusions on one side of the device as illustrated in FIG. 21.

In some embodiments of the present invention as disclosed herein, a stent or other metallic device can be mounted on the inflatable part of the device, so that in some embodiments, the stent can be deployed at the same time as when the inflatable chamber is inflated, or later on after inflating the device as illustrated in FIG. 24 in panel A, and B.

In some embodiments of the present invention as disclosed herein, the device may have one or more main inflatable chamber, and one or more side inflatable chambers. All of these chambers can be inflated at the same pressure with the same material (such as gas and/or liquid), or with different material and/or different pressures.

In some embodiments of the present invention as disclosed herein, the device can come in different lengths and different sizes, so it can be used in various indications, for example smaller sizes with thinner diameter for vascular indications, while larger sizes for gastrointestinal indications.

In some embodiments of the present invention as disclosed herein, inflatable chambers can come in different sizes to match the chamber, vessel, channel, aperture that needs to be dilated.

In some embodiments of the present invention as disclosed herein, minimum and maximal width and/or maximal and minimal length of each inflatable chamber can be predefined, refer to FIG. 20.

In some embodiments of the present invention as disclosed herein, there can one or more guidewire channels inside the device.

In some embodiments of the present invention as disclosed herein, the exterior part of the inflatable chamber may have convex or concave segments to enable better application force when the inflatable chamber is inflated as illustrated in FIG. 25.

In some embodiments of the present invention as disclosed herein, the inflation channel(s) can be connected to one or more pressure monitor through air and/or fluid tight channels that can be connected through inflation hub(s). Pressure sensor can be attached to the inflation channel and/or inflation port, directly or indirectly through a gas and/or liquid tight connection, and it is used to accurately monitor internal pressure of the main and side inflatable chamber(s), and to lower risk of rupture of the inflatable chambers if pressure is significantly elevated beyond what is manufacturer recommended based on testing of the device. In addition to that, pre-defined maximal inflation volume and/or inflating pressure may be defined for each of the inflatable chamber to lower risk of rupture. Limited pressure can be used, which can be defined experimentally to help avoid rupture of the inflatable chamber. It may range from 0.01 atmospheric pressure up to 500 atmospheric pressure.

In some embodiments of the present invention as disclosed herein, one or more Radio-opaque marker can be built, attached, or fused into the one or more of the walls of any of the main inflatable chambers, protrusions, side inflatable chambers, and/or catheter's shaft, which can be used to define the orientation of the catheter when it is rotated. The shape of the maker can be variable. This can be helpful in also defining the side of the protrusions or side inflatable chambers when the device is asymmetric. It also can be used to define the location and the extent of inflatable chamber etc. as illustrated in FIG. 26.

In some embodiments of the present invention as disclosed herein, the inflatable chamber, side inflatable chamber(s) and/or protrusions can have segments covered with metal, metallic blades, other cutting elements, or form various shapes. These metallic components can be embedded, attached or fused to the various segments of the inflatable chamber(s), side chambers and/or protrusions as illustrated in FIG. 27 in panel A, and B.

In some embodiments of the present invention as disclosed herein, the inflatable chambers' side chambers, protrusions can be impregnated or covered with medications that can help in preventing inflammation, scarring, anticoagulation etc.

In some embodiments of the present invention as disclosed herein, the device may have one or more Radio-opaque markers on or more sides of the inflatable chamber, protrusion(s), side Inflatable chamber, the shaft or anywhere along the length of the device. This radio-opaque marker can be useful to define the orientation of the balloon, so the operator will know which side is which. This can be helpful in using asymmetric devices.

In some embodiments of the present invention as disclosed herein, shaped balloon' technology can be used for coronary vascular diseases, peripheral vascular disease, venous diseases, gastrointestinal disease, urinary diseases and other medical illnesses.

In some embodiments of the present invention as disclosed herein, the new designed balloons can be used as a replacement of current balloon used in any available or future balloon angioplasty or inflatable medical devices.

In some embodiments of the present invention as disclosed herein, the shape and/or the size of any of the following: main inflatable chamber(s), side inflatable chamber(s), protrusions can change based on the volume or gas and/or liquid used to inflate the device.

In an embodiment of the present invention, it provides an inflatable device, the inflatable device comprises: one or more shafts; one or more shaft tips; one or more inflatable chambers; one or more inflation channels; one or more guidewire channels; one or more guidewire ports; one or more stiffening wires; and one or more inflation ports, wherein the inflatable device is shaped to treat the narrowing of fluid-carrying conduits or to treat the expansion of blockages in fluid-carrying conduits, wherein the shafts provide the main structure or skeleton of the inflatable device, wherein the shafts are made from a flexible material, wherein the shafts are non-inflatable, wherein the shafts have one or more ports that are connected to the different channels inside the shaft, wherein the said ports in the shafts are connected to inflation devices, syringes, and manometers to measure pressure, wherein the one or more inflation channels are located inside one of the shafts of the inflatable device and run along the entire length or part of the said one shaft of the device, wherein the inflation channels are used for inflating the inflatable chambers, wherein the inflation channels are air-tight and fluid-tight to limit leakage, wherein the inflation channels are connected to the inflation ports on one side and the inflatable chambers on the other side, wherein the one or more guidewire channels are located inside one of the shafts of the inflatable device and and run along the entire length or part of the said one shaft of the device, wherein the guidewire channels are associated with the inflation channels in a configuration selected from a group of configurations comprising a configuration where the guide wire channels run in the center of the shaft to the tip of the inflatable device, with the inflation channels surrounding the guide wire channels, and a configuration where the guide wire channels run parallel and/or adjacent to the inflation channels inside the shafts of the inflatable device, wherein the guidewire channels are used for passing the one or more guidewires inside the inflatable device, wherein the guidewire channels are connected to guidewire ports that are attached to one of the shafts of the inflatable device, wherein the inflatable chambers in the inflation device are the ones that treat the narrowing of fluid-carrying conduits or provide better expansion of blockages in fluid-carrying conduits, wherein the inflatable chambers are of a non-cylindrical shape or are non-circular in cross-section resulting in the inflatable device having a non-cylindrical shape or a non-circular cross-section, wherein the inflatable chambers are attached to the shaft of the inflatable device with the walls of the inflatable chambers partially fused to the shaft and connected to the inflation channels, this attachment and connection is air-tight and fluid-tight to limit leakage, wherein the inflatable device has variable maximum and minimum length, width, and/or depth with each of the one or more inflatable chambers having variable maximum and minimum length, width, and/or depth when inflated and deflated for different sizes of fluid-carrying conduits, wherein the inflatable chambers have walls that are made of materials selected from a group comprising polyvinyl chloride, polyethylene, polyethylene terephthalate, polyolefin copolymers, nylon derivatives, nylon-reinforced polyurethane, co-extruded copolymers, metallic elements, and alloys, wherein the walls of the inflatable chambers are formed from one or more layers of the same or different materials to enable shaping of the inflatable device, wherein the said materials are infused, attached, fused, or impregnated in the walls of the inflatable chamber for added functionality, wherein the one or more inflatable chambers have a non-cylindrical shape either when inflated, deflated, or both, wherein the non-cylindrical shape is selected from a group comprising oblong, oval, star-like with rounded edges, and triangular with rounded edges shape as viewed from the X, Y, and/or Z axis of one of the shafts of the device, wherein the inflatable chambers are angulated either when inflated, deflated, or both, compared to one of the shafts of the device and/or one of the guidewire channels, and the angle is selected from a group ranging between 0°- and 359°-degrees angle, wherein the inflatable chambers are selected from the group comprising symmetric and asymmetric to one of the shafts of the device and/or one of the guidewire channels along the X, Y, and/or Z axis of one of the shafts of the device leading to the shafts and/or guidewire channels not being in the centre of the inflatable chambers, wherein the inflatable chambers optionally have convex or concave segments on the exterior parts to enable better application of force when the inflatable chamber is inflated, wherein the guidewire channels are selected from a group of conformations comprising being on the side of the inflation channels adjected to the wall of the inflatable chambers with zero width between the wall of the inflatable channels and the guidewire channels and being attached to the outside of the inflation channel walls, wherein the one or more inflatable chambers are inflated in a manner selected from inflation manners selected from a group comprising an inflation manner where the one or more inflatable chambers are inflated at the same pressure with the same inflation agent, and an inflation manner where the one or more inflatable chambers are inflated at different pressures and/or with different inflation agents, wherein the inflation agent is a gas and/or a liquid, wherein the inflation channels are optionally connected through air-tight and fluid-tight means to limit leakage to one or more pressure monitors that are connected through inflation hubs to accurately monitor the internal pressure of the inflatable chambers to lower the risk of rupture of the inflatable chambers, wherein the inflatable chambers are folded around the shaft of the inflatable device when the device is deflated to minimize the overall diameter of the device and to enable insertion of the device in smaller fluid-carrying conduits, and wherein the inflatable chambers achieve better distribution of the force being applied and allow the use of a lower nominal pressure of the inflatable device to reduce acute complications.

In another embodiment of the present invention providing the inflatable device as disclosed herein, wherein the inflatable device optionally further comprises one or more protrusions, and wherein the protrusions are bulging of the periphery of one of the one or more inflatable chambers and the wall of the protrusion is contiguous with the wall of the inflatable chamber of which it is bulging out to allow inflation at the same time.

In another embodiment of the present invention providing the inflatable device as disclosed herein, wherein the inflatable device is a medical device to treat the narrowing of the fluid-carrying conduits or to treat the expansion of blockages in the fluid-carrying conduits in the body of an animal, wherein the fluid-carrying conduits include blood vessels, cardiac and non-cardiac chambers, and channels in the body of the animal, wherein the inflatable device is impregnated or covered with medications to help prevent inflammation, scarring, anticoagulation, and wherein the animal is a mammal.

In another embodiment of the present invention providing the inflatable device as disclosed herein, wherein the implantable device is a non-medical device to treat the narrowing of the fluid-carrying conduits or to treat the expansion of blockages in fluid-carrying conduits, wherein the fluid-carrying conduits include a tube, channel, conduit, or duct, wherein the fluid-carrying conduits regulate the flow of a liquid through said tube, channel, conduit, or duct.

In another embodiment of the present invention providing the inflatable device as disclosed herein, wherein the inflatable device has two or more inflatable chambers, wherein the inflation device has one main inflatable chamber among the one or more inflatable chambers and the other inflatable chambers are side inflatable chambers, wherein the side inflatable chambers are the ones with a wall that partially or completely separates them from the main inflatable chamber, wherein the inflatable device has a configuration selected from a group consisting of a configuration where the main inflatable chamber and side inflatable chambers have their own, separate inflatable channels and another configuration where the side inflatable chambers share an inflatable channel with the main inflatable chamber, wherein the inflation channels are connected to one or more outside inflation hubs, and wherein the inflation channels have no, partial or complete overlap between themselves along the X, Y, and/or Z axis of one of the shafts of the device.

In another embodiment of the present invention providing the inflatable device as disclosed herein, wherein the inflatable device has two or more inflatable chambers, wherein the inflation device has two or more main inflatable chambers among the one or more inflatable chambers and the remaining other inflatable chambers are side inflatable chambers, wherein the side inflatable chambers are the ones with a wall that partially or completely separates them from the main inflatable chambers, wherein the main inflatable chambers have similar shape and size, or they have different shapes and size, which can follow a certain pattern to the shaft of the device, wherein the inflatable device has a configuration selected from a group consisting of a configuration where the main inflatable chambers and side inflatable chambers have their own, separate inflatable channels and another configuration where the side inflatable chambers share an inflatable channel with one or more the main inflatable chambers, wherein the inflation channels are connected to one or more outside inflation hubs, and wherein the inflation channels have no, partial or complete overlap between themselves along the X, Y, and/or Z axis of one of the shafts of the device.

In another embodiment of the present invention providing the inflatable device as disclosed herein, wherein the inflatable device has two or more inflatable chambers, wherein the inflation device has one or more main inflatable chambers among the one or more inflatable chambers and the remaining other inflatable chambers are side inflatable chambers, wherein the one or more main inflatable chambers are encased in a larger inflatable chamber that is filled through its own, separate inflation channel, and/or a common inflation channel, wherein the larger inflatable chamber encasing the other inflatable chambers has different shapes for different functionality, wherein the inflation channels are connected to one or more outside inflation hubs, and wherein the inflation channels have no, partial or complete overlap between themselves along the X, Y, and/or Z axis of one of the shafts of the device.

In another embodiment of the present invention providing the inflatable device as disclosed herein, wherein the inflatable device has two or more inflatable chambers, wherein the inflation device has one or more main inflatable chambers among the one or more inflatable chambers and the remaining other inflatable chambers are side inflatable chambers, wherein the one or more main inflatable chambers are encased in a larger non-inflatable chamber that is filled through its own, separate inflation channel, and/or a common inflation channel, wherein the larger inflatable chamber encasing the other inflatable chambers has different shapes for different functionality, wherein the inflation channels are connected to one or more outside inflation hubs, and wherein the inflation channels have no, partial or complete overlap between themselves along the X, Y, and/or Z axis of one of the shafts of the device.

In another embodiment of the present invention providing the inflatable device as disclosed herein, wherein the inflatable device has two or more inflatable chambers, wherein the inflation device has one or more main inflatable chambers among the one or more inflatable chambers and the remaining other inflatable chambers are side inflatable chambers, wherein the inflatable device has a configuration selected from a group consisting of a configuration where the one or more main inflatable chambers run parallel to each other along the shaft of the device, and another configuration where the one or more main inflatable chambers run serially along the length of the shaft of the device, wherein the one or more main inflatable chambers is filled through its own, separate inflation channel, and/or a common inflation channel, wherein the inflation channels are connected to one or more outside inflation hubs, and wherein the inflation channels have no, partial or complete overlap between themselves along the X, Y, and/or Z axis of one of the shafts of the device.

In another embodiment of the present invention providing the inflatable device as disclosed herein, wherein the inflatable device has a stent or other metallic device mounted on the inflatable part of the device, wherein the stent or the other metallic device can be deployed at the same time as when the inflatable chamber is inflated, or later on after inflating the device.

In an embodiment of the present invention it provides an inflatable device, the inflatable device comprises: one or more shafts; one or more shaft tips; one or more main inflatable chambers; one or more side inflatable chambers; one or more inflation channels; one or more protrusions; one or more guidewire channels; one or more guidewire ports; one or more stiffening wires; and one or more inflation ports, wherein the inflatable device is shaped to treat the narrowing of fluid-carrying conduits or to treat the expansion of blockages in fluid-carrying conduits, wherein the shafts provide the main structure or skeleton of the inflatable device, wherein the shafts are made from a flexible material, wherein the shafts are non-inflatable, wherein the shafts have one or more ports that are connected to the different channels inside the shaft, wherein the said ports in the shafts are connected to inflation devices, syringes, and manometers to measure pressure, wherein the one or more inflation channels are located inside one of the shafts of the inflatable device and run along the entire length or part of the said one shaft of the device, wherein the inflation channels are used for inflating the inflatable chambers, wherein the inflation channels are air-tight and fluid-tight to limit leakage, wherein the inflation channels are connected to the inflation ports on one side and the inflatable chambers on the other side, wherein the protrusions are bulging out of the periphery of one of the one or more main inflatable chambers and the wall of the protrusion is contiguous with the wall of the main inflatable chamber of which it is bulging out to allow inflation at the same time, wherein the one or more protrusions and the one or more side inflatable chambers are present in a configuration of the inflatable device selected from a group comprising a configuration when the device is deflated, a configuration when the device is inflated, and a configuration when they are present when the device is inflated and deflated, wherein the one or more guidewire channels are located inside one of the shafts of the inflatable device and and run along the entire length or part of the said one shaft of the device, wherein the guidewire channels are associated with the inflation channels in a configuration selected from a group of configurations comprising a configuration where the guide wire channels run in the center of the shaft to the tip of the inflatable device, with the inflation channels surrounding the guide wire channels, and a configuration where the guide wire channels run parallel and/or adjacent to the inflation channels inside the shafts of the inflatable device, wherein the guidewire channels are used for passing the one or more guidewires inside the inflatable device, wherein the guidewire channels are connected to guidewire ports that are attached to one of the shafts of the inflatable device, wherein the inflatable chambers in the inflation device are the ones that treat the narrowing of fluid-carrying conduits or provide better expansion of blockages in fluid-carrying conduits, wherein the inflatable chambers are of a non-cylindrical shape or are non-circular in cross-section resulting in the inflatable device having a non-cylindrical shape or a non-circular cross-section, wherein the inflatable chambers are attached to the shaft of the inflatable device with the walls of the inflatable chambers partially fused to the shaft and connected to the inflation channels, this attachment and connection is air-tight and fluid-tight to limit leakage, wherein the inflatable device has variable maximum and minimum length, width, and/or depth with each of the one or more inflatable chambers having variable maximum and minimum length, width, and/or depth when inflated and deflated for different sizes of fluid-carrying conduits, wherein the inflatable chambers are folded around the shaft of the inflatable device when the device is deflated to minimize the overall diameter of the device and to enable insertion of the device in smaller fluid-carrying conduits, wherein the inflatable chambers achieve better distribution of the force being applied and allow the use of a lower nominal pressure of the inflatable device to reduce acute complications, wherein the one or more main inflatable chambers are attached to the shafts of the inflatable device and additional one or more side inflatable chambers are associated in a configuration selected from a group of configurations comprising a configuration where the one or more side inflatable chambers are attached to, fused to, or located adjacent to the outer part of the one or more main inflatable chambers, and a configuration where the one or more side inflatable chambers is built into the walls of one of the one or more main inflatable chambers, wherein the additional side inflatable chambers are associated in a configuration selected from a group of configurations comprising a configuration where the one or more side inflatable chambers have their own, separate inflation channels that run through the shaft of the device, and a configuration where the one or more side inflatable chambers are attached to a shared inflation channel, which is shared with the inflation channel that is connected to one of the one or more main inflatable chambers, wherein the inflation channels have a configuration selected from a group of configurations comprising a configuration where the inflation channels are connected to their own, separate inflation port, and a configuration where two or more inflation channels have a shared inflation port, wherein the inflatable chambers have walls that are made of materials selected from a group comprising polyvinyl chloride, polyethylene, polyethylene terephthalate, polyolefin copolymers, nylon derivatives, nylon-reinforced polyurethane, co-extruded copolymers, metallic elements, and alloys, wherein the walls of the inflatable chambers are formed from one or more layers of the same or different materials to enable shaping of the inflatable device, wherein the said materials are infused, attached, fused, or impregnated in the walls of the inflatable chamber for added functionality, wherein the one or more inflatable chambers have a non-cylindrical shape either when inflated, deflated, or both, wherein the non-cylindrical shape is selected from a group comprising oblong, oval, star-like with rounded edges, and triangular with rounded edges shape as viewed from the X, Y, and/or Z axis of one of the shafts of the device, wherein the inflatable chambers are angulated either when inflated, deflated, or both, compared to one of the shafts of the device and/or one of the guidewire channels, and the angle is selected from a group ranging between 0°- and 359°-degrees angle, wherein the inflatable chambers are selected from the group comprising symmetric and asymmetric to one of the shafts of the device and/or one of the guidewire channels along the X, Y, and/or Z axis of one of the shafts of the device leading to the shafts and/or guidewire channels not being in the centre of the inflatable chambers, wherein the inflatable chambers optionally have convex or concave segments on the exterior parts to enable better application of force when the inflatable chamber is inflated, wherein the guidewire channels are selected from a group of conformations comprising being on the side of the inflation channels adjected to the wall of the inflatable chambers with zero width between the wall of the inflatable channels and the guidewire channels and being attached to the outside of the inflation channel walls, wherein the one or more inflatable chambers are inflated in a manner selected from inflation manners selected from a group comprising an inflation manner where the one or more inflatable chambers are inflated at the same pressure with the same inflation agent, and an inflation manner where the one or more inflatable chambers are inflated at different pressures and/or with different inflation agents, wherein the inflation agent is a gas and/or a liquid, wherein the inflation channels are optionally connected through air-tight and fluid-tight means to limit leakage to one or more pressure monitors that are connected through inflation hubs to accurately monitor the internal pressure of the inflatable chambers to lower the risk of rupture of the inflatable chambers, wherein the inflatable device is impregnated or covered with medications to help prevent inflammation, scarring, anticoagulation, wherein the inflatable device is selected from a group comprising a medical device and a non-medical device, wherein the medical device is used to treat the narrowing of the fluid-carrying conduits or to treat the expansion of blockages in the fluid-carrying conduits in the body of an animal, wherein the fluid-carrying conduits include blood vessels, cardiac and non-cardiac chambers, and channels in the body of the animal, wherein the animal is a mammal, and wherein the non-medical device is used to treat the narrowing of the fluid-carrying conduits or to treat the expansion of blockages in fluid-carrying conduits, wherein the fluid-carrying conduits include a tube, channel, conduit, or duct, wherein the fluid-carrying conduits regulate the flow of a liquid through said tube, channel, conduit, or duct.

In another embodiment of the present invention providing the inflatable device as disclosed herein, wherein the one or more protrusions and/or one or more side inflatable chambers are formed by making a molded inflatable chamber with a shaped profile, wherein the shaped profile is a non-cylindrical shape or non-circular in cross-section resulting in the inflatable device having a non-cylindrical shape or a non-circular cross-section, wherein the protrusions and/or the side inflatable chambers have a round, oval, finger-like, irregular or any other shape, wherein the protrusions and/or the side inflatable chambers are selected from a group of configurations where they are similar in size and shape, and they are variable in size and shape, wherein the size and shape variability follows a pattern along the X, Y, and/or Z axis of one of the shafts of the device, and wherein the protrusions and side inflatable chambers have varied maximal and minimal width, length, location, orientation along the X, Y, and/or Z axis of one of the shafts of the device.

In another embodiment of the present invention providing the inflatable device as disclosed herein, wherein the one or more protrusions have walls with different physical properties in some parts of the protrusions or the entire protrusions to the main inflatable chamber from the periphery of which they are bulging out of, wherein the different physical properties include thickness, density of the walls of the protrusions, and number of layers that the walls of the protrusions form, wherein the walls of the protrusions have a different number of layers forming the walls to the other parts of the main inflatable chamber from the periphery of which they are bulging out of, wherein the walls of the protrusions have a different composition to the other parts of the main inflatable chamber from the periphery of which they are bulging out of, wherein the one or more protrusions have walls with different mechanical properties in some parts of the protrusions or the entire protrusions to the main inflatable chamber from the periphery of which they are bulging out of, wherein the different physical properties include elasticity, and strength, wherein the protrusions and the main inflatable chamber from the periphery of which they are bulging out of have different expansion properties to allow flexible and independent, semi-flexible and independent, semi-independent, or linked movement between the protrusions and the main inflatable chamber from the periphery of which they are bulging out of owing to the differences in their materials, physical properties, mechanical properties, wherein the said differences allow application of a higher pressure per surface area and/or higher sheer stress to an area of the fluid-carrying conduits when the protrusion in the inflatable device are inflated owing to differences in inflation and expansion between the protrusions and the main inflatable chambers, and at the same time, the said differences minimize the pressure being applied to the other areas of the fluid-carrying conduits, and improve the distribution of force being applied to better dilate the fluid-carrying conduits, wherein the area of the fluid-carrying conduits is a diseased, affected, or targeted area which is to be treated for the narrowing of the fluid-carrying conduits or for the expansion of blockages in the fluid-carrying conduits, and the other areas of the fluid-carrying conduits are normal, unaffected, or non-target areas for narrowing or expansion of the fluid-carrying conduits, wherein the protrusions have a pattern of distribution of their location, size, and/or shape along the perimeter and/or length of the main inflatable chamber from the periphery of which they are bulging out of, wherein the protrusions and the main inflatable chamber from the periphery of which they are bulging out expand in a manner selected from a group comprising a manner where the protrusions and the main inflatable chamber expand at the same speed, and a manner where the protrusions and the main inflatable chamber expand at varying speeds owing to the protrusions being more receptive or more resistant than the other parts of the main inflatable chamber to inflation due to the different physical or mechanical properties of the walls of the protrusions to the other parts of the main inflatable chamber.

In another embodiment of the present invention providing the inflatable device as disclosed herein, wherein the one or more side inflatable chambers have walls with different physical properties in some parts of the side inflatable chambers or the entire side inflatable chambers to the main inflatable chamber they are attached to, wherein the different physical properties include thickness, density of the walls of the side inflatable chambers, and number of layers that the walls of the side inflatable chambers form, wherein the walls of the side inflatable chambers have a different number of layers forming the walls to the other parts of the main inflatable chamber they are attached to, wherein the walls of the side inflatable chambers have a different composition to the other parts of the main inflatable chamber they are attached to, wherein the one or more side inflatable chambers have walls with different mechanical properties in some parts of the side inflatable chambers or the entire side inflatable chambers to the main inflatable chamber they are attached to, wherein the different physical properties include elasticity, and strength, wherein the side inflatable chambers and the main inflatable chamber they are attached to have different expansion properties to allow flexible and independent, semi-flexible and independent, semi-independent, or linked movement between the side inflatable chambers and the main inflatable chamber they are attached to owing to the differences in their materials, physical properties, mechanical properties, wherein the side inflatable chambers and the main inflatable chamber to which they are associated expand in a manner selected from a group comprising a manner where the side inflatable chambers and the main inflatable chamber expand at the same speed, and a manner where the side inflatable chambers and the main inflatable chamber expand at varying speeds owing to the side inflatable chambers being more receptive or more resistant than the other parts of the main inflatable chamber to inflation due to the different physical or mechanical properties of the walls of the side inflatable chambers to the other parts of the main inflatable chamber.

In another embodiment of the present invention providing the inflatable device as disclosed herein, wherein the inflatable device has a stent or other metallic device mounted on the inflatable part of the device, wherein the stent or the other metallic device can be deployed at the same time as when the inflatable chamber is inflated, or later on after inflating the device.

In another embodiment of the present invention providing the inflatable device as disclosed herein, wherein the shafts of the inflatable device, the main inflatable chambers, the side inflatable chambers, and/or the protrusions optionally have one or more radio-opaque markers built, attached, or fused into the walls of the shafts of the inflatable device, the main inflatable chambers, the side inflatable chambers, and/or the protrusions, for defining the orientation of the shafts of the inflatable device, main inflatable chambers, the side inflatable chambers, and/or the protrusions when the inflatable device is rotated and to define the location and extent of the shafts of the inflatable device, main inflatable chambers, the side inflatable chambers, and/or the protrusions, and wherein the shape of the marker is variable.

In another embodiment of the present invention providing the inflatable device as disclosed herein, wherein the main inflatable chambers, the side inflatable chambers, and/or the protrusions optionally have segments covered with one or more covering materials, wherein the covering materials comprise metal, metallic blades, and other cutting elements, wherein the covering materials aid the main inflatable chambers, the side inflatable chambers, and/or the protrusions and in turn, the inflatable device form various shapes, and wherein the covering materials are embedded, attached or fused to the segments of the main inflatable chambers, the side inflatable chambers, and/or the protrusions.

In an embodiment of the present invention, it provides an inflatable device, the inflatable device comprises: one or more shafts; one or more shaft tips; one or more main inflatable chambers; one or more side inflatable chambers; one or more inflation channels; one or more protrusions; one or more accessories; one or more guidewire channels; one or more guidewire ports; one or more stiffening wires; and one or more inflation ports, wherein the inflatable device is shaped to treat the narrowing of fluid-carrying conduits or to treat the expansion of blockages in fluid-carrying conduits, wherein the shafts provide the main structure or skeleton of the inflatable device, wherein the shafts are made from a flexible material, wherein the shafts are non-inflatable, wherein the shafts have one or more ports that are connected to the different channels inside the shaft, wherein the said ports in the shafts are connected to inflation devices, syringes, and manometers to measure pressure, wherein the one or more inflation channels are located inside one of the shafts of the inflatable device and run along the entire length or part of the said one shaft of the device, wherein the inflation channels are used for inflating the inflatable chambers, wherein the inflation channels are air-tight and fluid-tight to limit leakage, wherein the inflation channels are connected to the inflation ports on one side and the inflatable chambers on the other side, wherein the protrusions are bulging out of the periphery of one of the one or more main inflatable chambers and the wall of the protrusion is contiguous with the wall of the main inflatable chamber of which it is bulging out to allow inflation at the same time, wherein the one or more protrusions and the one or more side inflatable chambers are present in a configuration of the inflatable device selected from a group comprising a configuration when the device is deflated, a configuration when the device is inflated, and a configuration when they are present when the device is inflated and deflated, wherein the one or more accessories include constrictive wires, lines, cylinders, and tubes, wherein the one or more accessories are selected from a group of configurations where they are made from the same materials entirely, and where they are made from different materials which follow a pattern, wherein the one or more accessories are made from materials that include non-flexible, semi-flexible material, memory metal, other metals, wherein the memory metal includes Nitinol, wherein the other metals include stainless steel, and titanium, wherein the one or more guidewire channels are located inside one of the shafts of the inflatable device and and run along the entire length or part of the said one shaft of the device, wherein the guidewire channels are associated with the inflation channels in a configuration selected from a group of configurations comprising a configuration where the guide wire channels run in the center of the shaft to the tip of the inflatable device, with the inflation channels surrounding the guide wire channels, and a configuration where the guide wire channels run parallel and/or adjacent to the inflation channels inside the shafts of the inflatable device, wherein the guidewire channels are used for passing the one or more guidewires inside the inflatable device, wherein the guidewire channels are connected to guidewire ports that are attached to one of the shafts of the inflatable device, wherein the inflatable chambers in the inflation device are the ones that treat the narrowing of fluid-carrying conduits or provide better expansion of blockages in fluid-carrying conduits, wherein the inflatable chambers are of a non-cylindrical shape or are non-circular in cross-section resulting in the inflatable device having a non-cylindrical shape or a non-circular cross-section, wherein the inflatable chambers are attached to the shaft of the inflatable device with the walls of the inflatable chambers partially fused to the shaft and connected to the inflation channels, this attachment and connection is air-tight and fluid-tight to limit leakage, wherein the inflatable device has variable maximum and minimum length, width, and/or depth with each of the one or more inflatable chambers having variable maximum and minimum length, width, and/or depth when inflated and deflated for different sizes of fluid-carrying conduits, wherein the inflatable chambers are folded around the shaft of the inflatable device when the device is deflated to minimize the overall diameter of the device and to enable insertion of the device in smaller fluid-carrying conduits, wherein the inflatable chambers achieve better distribution of the force being applied and allow the use of a lower nominal pressure of the inflatable device to reduce acute complications, wherein the inflatable chambers have walls that are made of materials selected from a group comprising polyvinyl chloride, polyethylene, polyethylene terephthalate, polyolefin copolymers, nylon derivatives, nylon-reinforced polyurethane, co-extruded copolymers, metallic elements, and alloys, wherein the walls of the inflatable chambers are formed from one or more layers of the same or different materials to enable shaping of the inflatable device, wherein the said materials are infused, attached, fused, or impregnated in the walls of the inflatable chamber for added functionality, wherein the one or more inflatable chambers have a non-cylindrical shape either when inflated, deflated, or both, wherein the non-cylindrical shape is selected from a group comprising oblong, oval, star-like with rounded edges, and triangular with rounded edges shape as viewed from the X, Y, and/or Z axis of one of the shafts of the device, wherein the inflatable chambers are angulated either when inflated, deflated, or both, compared to one of the shafts of the device and/or one of the guidewire channels, and the angle is selected from a group ranging between 0°- and 359°-degrees angle, wherein the inflatable chambers are selected from the group comprising symmetric and asymmetric to one of the shafts of the device and/or one of the guidewire channels along the X, Y, and/or Z axis of one of the shafts of the device leading to the shafts and/or guidewire channels not being in the centre of the inflatable chambers, wherein the inflatable chambers optionally have convex or concave segments on the exterior parts to enable better application of force when the inflatable chamber is inflated, wherein the guidewire channels are selected from a group of conformations comprising being on the side of the inflation channels adjected to the wall of the inflatable chambers with zero width between the wall of the inflatable channels and the guidewire channels and being attached to the outside of the inflation channel walls, wherein the one or more inflatable chambers are inflated in a manner selected from inflation manners selected from a group comprising an inflation manner where the one or more inflatable chambers are inflated at the same pressure with the same inflation agent, and an inflation manner where the one or more inflatable chambers are inflated at different pressures and/or with different inflation agents, wherein the inflation agent is a gas and/or a liquid, wherein the inflation channels are optionally connected through air-tight and fluid-tight means to limit leakage to one or more pressure monitors that are connected through inflation hubs to accurately monitor the internal pressure of the inflatable chambers to lower the risk of rupture of the inflatable chambers, wherein the inflatable device is impregnated or covered with medications to help prevent inflammation, scarring, anticoagulation, wherein the inflatable device is selected from a group comprising a medical device and a non-medical device, wherein the medical device is used to treat the narrowing of the fluid-carrying conduits or to treat the expansion of blockages in the fluid-carrying conduits in the body of an animal, wherein the fluid-carrying conduits include blood vessels, cardiac and non-cardiac chambers, and channels in the body of the animal, wherein the animal is a mammal, and wherein the non-medical device is used to treat the narrowing of the fluid-carrying conduits or to treat the expansion of blockages in fluid-carrying conduits, wherein the fluid-carrying conduits include a tube, channel, conduit, or duct, wherein the fluid-carrying conduits regulate the flow of a liquid through said tube, channel, conduit, or duct.

In another embodiment of the present invention providing the inflatable device as disclosed herein, wherein the one or more accessories are associated with the inflatable device in a manner selected from configurations where they are embedded, fused, and/or attached to the walls of the main inflatable chamber and/or the walls of the side inflatable chambers walls, and/or the walls of the protrusions, and where they are located on the outside of the walls of the main inflatable chamber and/or the walls of the side inflatable chambers walls, and/or the walls of the protrusions, and wherein the one or more accessories are associated with the inflatable device in the manner that upon inflation of the inflatable device, the presence of the one or more accessories results in restriction of the expansion of the main inflatable chamber and/or the side inflatable chambers, and/or the protrusions, wherein the restriction in expansion is in a pattern that creates a shaped inflatable device required to treat the narrowing of the fluid-carrying conduits or to treat the expansion of blockages in fluid-carrying conduits, wherein the pattern includes configurations selected from a group comprising a configuration where the one or more accessories are located at one or more locations along the X, Y, and/or Z axis of the shafts of inflatable device, and/or a configuration where the one or more accessories are distributed at and around one or more parts of the main inflatable chamber and/or the side inflatable chambers, and/or the protrusions, wherein said configurations are used to create the shaped inflatable device required to treat the narrowing of the fluid-carrying conduits or to treat the expansion of blockages in fluid-carrying conduits.

In another embodiment of the present invention providing the inflatable device as disclosed herein, wherein the inflatable device has a stent or other metallic device mounted on the inflatable part of the device, wherein the stent or the other metallic device can be deployed at the same time as when the inflatable chamber is inflated, or later on after inflating the device.

In an embodiment of the present invention, it provides a method for treating the narrowing of the fluid-carrying conduits in the body of an animal, the method comprising the steps of: obtaining an inflatable device as disclosed in the present invention hereinabove, sterilizing the inflatable device, inserting and introducing the inflatable device through an orifice or a small hole into the body of the animal, advancing the inflatable device inside a fluid-carrying conduit in the body of the animal towards a target area using a guidewire under fluoroscopy guidance, verifying that the inflatable device is in the correct position at and/or around the target area, inflating one or more inflatable chambers in the inflatable device using a gas and/or a liquid to a certain pressure and/or to a certain volume to achieve dilation of the target area in a target organ in the body of the animal while minimizing the risk of a rupture of the one or more inflatable chambers for treating the narrowing of the fluid-carrying conduits in the body of an animal, wherein the inflatable device is a medical device, wherein the fluid-carrying conduits include blood vessels, cardiac and non-cardiac chambers, and channels in the body of the animal, wherein the animal is a mammal, wherein the inflating is done after verifying by injecting a progressive amount of a gas and/or a liquid using one or more inflation devices attached to one or more inflation channels through one or more inflation ports to inflate one or more inflatable chambers, wherein the inflating results in expansion of the one or more inflatable chambers under fluoroscopy guidance until a certain pressure is achieved and/or maximal volume is injected inside the one or more inflatable chambers through the inflatable device including a delivery catheter and/or until the narrowing in the target fluid-carrying conduits is expanded to a certain degree, wherein inflating is done using a gas or a liquid selected from a group comprising ambient air, other types of gases and/or liquids and/or a combination thereof, wherein the inflating is stopped once the fluid-carrying conduits reach an expansion of optimal size and narrowing is treated or a certain maximum inflation volume or inflation pressure is achieved, wherein the inflating is done using a special inflation device which includes a syringe and the inflation set up has one or more pressure measuring devices and/or sensors and/or manometers, wherein one or more inflatable devices are optionally used in the inflation process, and wherein each of the one or more inflatable devices are connected to specific inflation channels inside the shafts of the inflatable devices that are connected to one or more inflatable chambers.

In an embodiment of the present invention, it provides a method for treating the narrowing of the fluid-carrying conduits in the body of an animal, the method comprising the steps of: obtaining an inflatable device as disclosed in the present invention hereinabove, obtaining an inflatable device as disclosed in the present invention hereinabove, sterilizing the inflatable device, inserting and introducing the inflatable device through an orifice or a small hole into the body of the animal, advancing the inflatable device inside a fluid-carrying conduit in the body of the animal towards a target area using a guidewire under fluoroscopy guidance, verifying that the inflatable device is in the correct position at and/or around the target area, inflating one or more inflatable chambers in the inflatable device using a gas and/or a liquid to a certain pressure and/or to a certain volume to achieve dilation of the target area in a target organ in the body of the animal while minimizing the risk of a rupture of the one or more inflatable chambers for treating the narrowing of the fluid-carrying conduits in the body of the animal, wherein the inflatable device is a medical device, wherein the fluid-carrying conduits include blood vessels, cardiac and non-cardiac chambers, and channels in the body of the animal, wherein the animal is a mammal, wherein the inflating is done after verifying by injecting a progressive amount of a gas and/or a liquid using one or more inflation devices attached to one or more inflation channels through one or more inflation ports to inflate one or more inflatable chambers, wherein the inflating results in expansion of the one or more inflatable chambers under fluoroscopy guidance until a certain pressure is achieved and/or maximal volume is injected inside the one or more inflatable chambers through the inflatable device including a delivery catheter and/or until the narrowing in the target fluid-carrying conduits is expanded to a certain degree, wherein inflating is done using a gas or a liquid selected from a group comprising ambient air, other types of gases and/or liquids and/or a combination thereof, wherein the inflating is stopped once the fluid-carrying conduits reach an expansion of optimal size and narrowing is treated or a certain maximum inflation volume or inflation pressure is achieved, wherein the inflating is done using a special inflation device which includes a syringe and the inflation set up has one or more pressure measuring devices and/or sensors and/or manometers, wherein one or more inflatable devices are optionally used in the inflation process, and wherein each of the one or more inflatable devices are connected to specific inflation channels inside the shafts of the inflatable devices that are connected to one or more inflatable chambers.

In an embodiment of the present invention, it provides a method for treating the narrowing of the fluid-carrying conduits or to treat the expansion of blockages in fluid-carrying conduits, the method comprising the steps of: obtaining an inflatable device as disclosed in the present invention hereinabove, obtaining an inflatable device as disclosed in the present invention hereinabove, sterilizing the inflatable device, inserting and introducing the inflatable device through an orifice or a small hole into the fluid-carrying conduits, advancing the inflatable device inside a fluid-carrying conduit towards a target area using a guidewire under fluoroscopy guidance, verifying that the inflatable device is in the correct position at and/or around the target area, inflating one or more inflatable chambers in the inflatable device using a gas and/or a liquid to a certain pressure and/or to a certain volume to achieve dilation of the target area in the fluid-carrying conduits while minimizing the risk of a rupture of the one or more inflatable chambers for treating the expansion of blockages in fluid-carrying conduits, wherein the inflatable device is a non-medical device, wherein the fluid-carrying conduits include a tube, channel, conduit, or duct, wherein the fluid-carrying conduits regulate the flow of a liquid through said tube, channel, conduit, or duct, wherein the inflating is done after verifying by injecting a progressive amount of a gas and/or a liquid using one or more inflation devices attached to one or more inflation channels through one or more inflation ports to inflate one or more inflatable chambers, wherein the inflating results in expansion of the one or more inflatable chambers under fluoroscopy guidance until a certain pressure is achieved and/or maximal volume is injected inside the one or more inflatable chambers through the inflatable device including a delivery catheter and/or until the narrowing in the target fluid-carrying conduits is expanded to a certain degree, wherein inflating is done using a gas or a liquid selected from a group comprising ambient air, other types of gases and/or liquids and/or a combination thereof, wherein the inflating is stopped once the fluid-carrying conduits reach an expansion of optimal size and narrowing is treated or a certain maximum inflation volume or inflation pressure is achieved, wherein the inflating is done using a special inflation device which includes a syringe and the inflation set up has one or more pressure measuring devices and/or sensors and/or manometers, wherein one or more inflatable devices are optionally used in the inflation process, and wherein each of the one or more inflatable devices are connected to specific inflation channels inside the shafts of the inflatable devices that are connected to one or more inflatable chambers.

The invention will be further explained by the following Examples, which are intended to be purely exemplary of the invention and should not be considered as limiting the invention in any way.

### EXAMPLES

The following example provides exemplary embodiments of the implantable devices and sealing halo units of the present invention.

### Example 1

In this example of the present invention, embodiments of the present invention are exemplified in the form of catheters as the inflatable device of the present invention, where two or more wires are attached to a catheter shaft as representative examples of the present invention, typically distal and proximal to the balloons, i.e., representative inflatable chambers of the present invention, along the Z axis of the catheter's shaft. These wires are made from the same material, typically metal such as stainless steel, Nitinol, etc.

In exemplary embodiments of the present invention, the segments of the balloon that are adjacent to each of these wires are optionally thinner or thicker when compared with the remainder of the balloon wall, these segments are also optionally made from a different material as compared with the remainder of the balloon wall, and/or these segments optionally have different mechanical or physical properties (for example, more or less compliant) when compared with the remainder of the balloon's wall.

In exemplary embodiments of the present invention, the segments of the balloon's wall are made to be thinner, where this change helps the adjacent wires stay in the grooves created by these segments when the balloon is inflated, and they also help in creating the shape of the balloon.

In FIG. 28, a cross-sectional view of one such embodiment of the present invention is exemplified, where wires (1) run adjacent to thinner segments of the balloon wall (2), thus creating grooves in the balloon when it is inflated, the remainder of the balloon wall is thicker (3) when compared with the segments that are adjacent to the wires (2). Also identified in the illustration are an inflation channel (6), an inflation hole (5) through which the balloon is inflated, and a wire channel (7).

In FIG. 29, a cross-sectional view of one such embodiment of the present invention is exemplified, where wires (1) are attached to the catheter's shaft (2). Also identified in the illustration are a balloon (4), wires attached to the catheter's shaft proximal (6) to the balloon and distal (5) to the balloon, the tip of the catheter (3), where wires create grooves in the balloon by restricting expansion certain parts of the balloon.

### Example 2

In this example of the present invention, embodiments of the present invention are exemplified in the form of catheters as the inflatable device of the present invention, where wires are embedded inside the balloon wall, thus restricting part of the balloon where they are embedded, and not restricting the other segments of the balloon. The free ends of the wires are optionally attached to the catheter's shaft proximal and distal to the balloon.

In FIG. 30, a cross-sectional view of one such embodiment of the present invention is exemplified, where wires (2) are embedded inside the balloon wall (3), thus restricting part of the balloon where they are embedded, and not restricting of other segments of the balloon (3). Also identified in the illustration are wires that are attached to the catheter's shaft (1), an inflation channel (5), and a wire channel (6).

In FIG. 31, a cross-sectional view of one such embodiment of the present invention is exemplified, where wires (3, 4) are embedded inside the balloon wall (2), thus restricting part of the balloon where they are embedded, and not restricting the other segments of the balloon. Also identified in the illustration is the tip of the catheter (8), wires that are attached to the catheter's shaft (1) proximally (5) and distally (6), part of the wires may be exposed (3, 4) and are not embedded inside the balloon. In the illustration shown further is a segment of the balloon (7) that is restricted by the embedded wire, while in another segment of the balloon (2) where the wire is not embedded, the balloon can expand more freely.

### Example 3

In this example of the present invention, embodiments of the present invention are exemplified in the form of catheters as the inflatable device of the present invention, where there are primary wires that are attached to the catheter's shaft proximal, and distal to the balloon, in addition to that there are one or more cross wires that connect between two or more primary wires, to minimize slippage of the wires over the balloon when the balloon is inflated. These wires are optionally attached at an angle that ranges from 0 degrees up to 360 degrees to the primary wires. Cross wires are optionally made from metal such as stainless steel, Nitinol, etc. Cross wires are optionally made from the same or different material as compared to primary wires.

In FIG. 32, a cross-sectional view of one such embodiment of the present invention is exemplified with cross wires, where primary wires (5, 8) are attached to the catheter's shaft, while cross wires (4, 6, 7) connect between the primary wires. Also identified in the illustration is the tip of the catheter (2).

In FIG. 33, a cross-sectional view of one such embodiment of the present invention is exemplified with cross wires, where primary wires (1, 3) are attached to the catheter's shaft proximally and distally (7, 4), while cross wires (2, 8) connect between the primary wires. Also identified in the illustration are the tip of the catheter (5), the catheter shaft (6), and the balloon (11). In the illustration shown further is the angle (9, 10) between the cross wire and the primary wire which can range from 0° to 360° degrees.

The above-mentioned embodiments can be used, individually or in any combination(s), as a part of any inflatable device or used as a modification to the device. These devices can be any inflatable medical device for treating blockages or functional non-medical devices that can be used in other industries such as the oil industry.

It will be apparent to those skilled in the art that various modifications and variations can be made in the practice of the present invention without departing from the scope or spirit of the invention. Other embodiments of the invention will be apparent to those skilled in the art from considering of the specification and practice of the invention. It is intended that the specification and examples be considered as exemplary only, with a true scope and spirit of the invention being indicated by the following claims.

What is claimed is:

1. An inflatable device, the inflatable device comprises:
one or more shafts;
one or more shaft tips;
one or more inflatable chambers;
one or more inflation channels;
one or more guidewire channels;
one or more guidewire ports;
one or more stiffening wires; and
one or more inflation ports,
wherein the inflatable device is shaped to treat the narrowing of fluid-carrying conduits or to treat the expansion of blockages in fluid-carrying conduits,
wherein the shafts provide the main structure or skeleton of the inflatable device,
wherein the shafts are made from a flexible material,
wherein the shafts are non-inflatable,
wherein the shafts have one or more ports that are connected to the different channels inside the shaft,
wherein the said ports in the shafts are connected to inflation devices, syringes, and manometers to measure pressure,
wherein the one or more inflation channels are located inside one of the shafts of the inflatable device and run along the entire length or part of the said one shaft of the device,
wherein the inflation channels are used for inflating the inflatable chambers,
wherein the inflation channels are air-tight and fluid-tight to limit leakage,
wherein the inflation channels are connected to the inflation ports on one side and the inflatable chambers on the other side,
wherein the one or more guidewire channels are located inside one of the shafts of the inflatable device and and run along the entire length or part of the said one shaft of the device,
wherein the guidewire channels are associated with the inflation channels in a configuration selected from a group of configurations comprising a configuration where the guide wire channels run in the center of the shaft to the tip of the inflatable device, with the inflation channels surrounding the guide wire channels, and a configuration where the guide wire channels run parallel and/or adjacent to the inflation channels inside the shafts of the inflatable device,
wherein the guidewire channels are used for passing the one or more guidewires inside the inflatable device,
wherein the guidewire channels are connected to guidewire ports that are attached to one of the shafts of the inflatable device,
wherein the inflatable chambers in the inflation device are the ones that treat the narrowing of fluid-carrying conduits or provide better expansion of blockages in fluid-carrying conduits,
wherein the inflatable chambers are of a non-cylindrical shape or are non-circular in cross-section resulting in the inflatable device having a non-cylindrical shape or a non-circular cross-section,
wherein the inflatable chambers are attached to the shaft of the inflatable device with the walls of the inflatable chambers partially fused to the shaft and connected to the inflation channels, this attachment and connection is air-tight and fluid-tight to limit leakage,
wherein the inflatable device has variable maximum and minimum length, width, and/or depth with each of the one or more inflatable chambers having variable maximum and minimum length, width, and/or depth when inflated and deflated for different sizes of fluid-carrying conduits,
wherein the inflatable chambers have walls that are made of materials selected from a group comprising polyvinyl chloride, polyethylene, polyethylene terephthalate, polyolefin copolymers, nylon derivatives, nylon-reinforced polyurethane, co-extruded copolymers, metallic elements, and alloys,
wherein the walls of the inflatable chambers are formed from one or more layers of the same or different materials to enable shaping of the inflatable device,
wherein the said materials are infused, attached, fused, or impregnated in the walls of the inflatable chamber for added functionality,
wherein the one or more inflatable chambers have a non-cylindrical shape either when inflated, deflated, or both,
wherein the non-cylindrical shape is selected from a group comprising oblong, oval, star-like with rounded edges, and triangular with rounded edges shape as viewed from the X, Y, and/or Z axis of one of the shafts of the device,
wherein the inflatable chambers are angulated either when inflated, deflated, or both, compared to one of the shafts of the device and/or one of the guidewire channels, and the angle is selected from a group ranging between 0°- and 359°-degrees angle,
wherein the inflatable chambers are selected from the group comprising symmetric and asymmetric to one of the shafts of the device and/or one of the guidewire channels along the X, Y, and/or Z axis of one of the shafts of the device leading to the shafts and/or guidewire channels not being in the centre of the inflatable chambers,
wherein the inflatable chambers optionally have convex or concave segments on the exterior parts to enable better application of force when the inflatable chamber is inflated,
wherein the guidewire channels are selected from a group of conformations comprising being on the side of the inflation channels adjected to the wall of the inflatable chambers with zero width between the wall of the inflatable channels and the guidewire channels and being attached to the outside of the inflation channel walls,
wherein the one or more inflatable chambers are inflated in a manner selected from inflation manners selected from a group comprising an inflation manner where the one or more inflatable chambers are inflated at the same pressure with the same inflation agent, and an inflation manner where the one or more inflatable chambers are inflated at different pressures and/or with different inflation agents,
wherein the inflation agent is a gas and/or a liquid,
wherein the inflation channels are optionally connected through air-tight and fluid-tight means to limit leakage to one or more pressure monitors that are connected through inflation hubs to accurately monitor the internal pressure of the inflatable chambers to lower the risk of rupture of the inflatable chambers,
wherein the inflatable chambers are folded around the shaft of the inflatable device when the device is deflated to minimize the overall diameter of the device and to enable insertion of the device in smaller fluid-carrying conduits, and
wherein the inflatable chambers achieve better distribution of the force being applied and allow the use of a lower nominal pressure of the inflatable device to reduce acute complications.

2. The inflatable device of claim 1, wherein the inflatable device optionally further comprises one or more protrusions, and wherein the protrusions are bulging of the periphery of one of the one or more inflatable chambers and the wall of the protrusion is contiguous with the wall of the inflatable chamber of which it is bulging out to allow inflation at the same time.

3. The inflatable device of claim 1, wherein the inflatable device is a medical device to treat the narrowing of the fluid-carrying conduits or to treat the expansion of blockages in the fluid-carrying conduits in the body of an animal, wherein the fluid-carrying conduits include blood vessels, cardiac and non-cardiac chambers, and channels in the body of the animal, wherein the inflatable device is impregnated or covered with medications to help prevent inflammation, scarring, anticoagulation, and wherein the animal is a mammal.

4. The inflatable device of claim 1, wherein the implantable device is a non-medical device to treat the narrowing of the fluid-carrying conduits or to treat the expansion of blockages in fluid-carrying conduits, wherein the fluid-carrying conduits include a tube, channel, conduit, or duct, wherein the fluid-carrying conduits regulate the flow of a liquid through said tube, channel, conduit, or duct.

5. The inflatable device of claim 1, wherein the inflatable device has two or more inflatable chambers, wherein the inflation device has one main inflatable chamber among the one or more inflatable chambers and the other inflatable chambers are side inflatable chambers, wherein the side inflatable chambers are the ones with a wall that partially or completely separates them from the main inflatable chamber, wherein the inflatable device has a configuration selected from a group consisting of a configuration where the main inflatable chamber and side inflatable chambers have their own, separate inflatable channels and another configuration where the side inflatable chambers share an inflatable channel with the main inflatable chamber, wherein the inflation channels are connected to one or more outside inflation hubs, and wherein the inflation channels have no, partial or complete overlap between themselves along the X, Y, and/or Z axis of one of the shafts of the device.

6. The inflatable device of claim 1, wherein the inflatable device has two or more inflatable chambers, wherein the inflation device has two or more main inflatable chambers among the one or more inflatable chambers and the remaining other inflatable chambers are side inflatable chambers, wherein the side inflatable chambers are the ones with a wall that partially or completely separates them from the main inflatable chambers, wherein the main inflatable chambers have similar shape and size, or they have different shapes and size, which can follow a certain pattern to the shaft of the device, wherein the inflatable device has a configuration selected from a group consisting of a configuration where the main inflatable chambers and side inflatable chambers have their own, separate inflatable channels and another configuration where the side inflatable chambers share an inflatable channel with one or more the main inflatable chambers, wherein the inflation channels are connected to one or more outside inflation hubs, and wherein the inflation channels have no, partial or complete overlap between themselves along the X, Y, and/or Z axis of one of the shafts of the device.

7. The inflatable device of claim 1, wherein the inflatable device has two or more inflatable chambers, wherein the inflation device has one or more main inflatable chambers among the one or more inflatable chambers and the remaining other inflatable chambers are side inflatable chambers, wherein the one or more main inflatable chambers are encased in a larger inflatable chamber that is filled through its own, separate inflation channel, and/or a common inflation channel, wherein the larger inflatable chamber encasing the other inflatable chambers has different shapes for different functionality, wherein the inflation channels are connected to one or more outside inflation hubs, and wherein the inflation channels have no, partial or complete overlap between themselves along the X, Y, and/or Z axis of one of the shafts of the device.

8. The inflatable device of claim 1, wherein the inflatable device has two or more inflatable chambers, wherein the inflation device has one or more main inflatable chambers among the one or more inflatable chambers and the remaining other inflatable chambers are side inflatable chambers, wherein the one or more main inflatable chambers are encased in a larger non-inflatable chamber that is filled through its own, separate inflation channel, and/or a common inflation channel, wherein the larger inflatable chamber encasing the other inflatable chambers has different shapes for different functionality, wherein the inflation channels are connected to one or more outside inflation hubs, and wherein the inflation channels have no, partial or complete overlap between themselves along the X, Y, and/or Z axis of one of the shafts of the device.

9. The inflatable device of claim 1, wherein the inflatable device has two or more inflatable chambers, wherein the inflation device has one or more main inflatable chambers among the one or more inflatable chambers and the remaining other inflatable chambers are side inflatable chambers, wherein the inflatable device has a configuration selected from a group consisting of a configuration where the one or more main inflatable chambers run parallel to each other along the shaft of the device, and another configuration where the one or more main inflatable chambers run serially along the length of the shaft of the device, wherein the one or more main inflatable chambers is filled through its own, separate inflation channel, and/or a common inflation channel, wherein the inflation channels are connected to one or more outside inflation hubs, and wherein the inflation channels have no, partial or complete overlap between themselves along the X, Y, and/or Z axis of one of the shafts of the device.

10. The inflatable device of claim 1, wherein the inflatable device has a stent or other metallic device mounted on the inflatable part of the device, wherein the stent or the other metallic device can be deployed at the same time as when the inflatable chamber is inflated, or later on after inflating the device.

11. An inflatable device, the inflatable device comprises:
one or more shafts;
one or more shaft tips;
one or more main inflatable chambers;
one or more side inflatable chambers;
one or more inflation channels;
one or more protrusions;
one or more guidewire channels;
one or more guidewire ports;
one or more stiffening wires; and
one or more inflation ports,
wherein the inflatable device is shaped to treat the narrowing of fluid-carrying conduits or to treat the expansion of blockages in fluid-carrying conduits,
wherein the shafts provide the main structure or skeleton of the inflatable device,
wherein the shafts are made from a flexible material,
wherein the shafts are non-inflatable,
wherein the shafts have one or more ports that are connected to the different channels inside the shaft,
wherein the said ports in the shafts are connected to inflation devices, syringes, and manometers to measure pressure,
wherein the one or more inflation channels are located inside one of the shafts of the inflatable device and run along the entire length or part of the said one shaft of the device,
wherein the inflation channels are used for inflating the inflatable chambers,
wherein the inflation channels are air-tight and fluid-tight to limit leakage,
wherein the inflation channels are connected to the inflation ports on one side and the inflatable chambers on the other side,
wherein the protrusions are bulging out of the periphery of one of the one or more main inflatable chambers and the wall of the protrusion is contiguous with the wall of the main inflatable chamber of which it is bulging out to allow inflation at the same time,
wherein the one or more protrusions and the one or more side inflatable chambers are present in a configuration of the inflatable device selected from a group comprising a configuration when the device is deflated, a configuration when the device is inflated, and a configuration when they are present when the device is inflated and deflated,
wherein the one or more guidewire channels are located inside one of the shafts of the inflatable device and and run along the entire length or part of the said one shaft of the device,
wherein the guidewire channels are associated with the inflation channels in a configuration selected from a group of configurations comprising a configuration where the guide wire channels run in the center of the shaft to the tip of the inflatable device, with the inflation channels surrounding the guide wire channels, and a configuration where the guide wire channels run parallel and/or adjacent to the inflation channels inside the shafts of the inflatable device,
wherein the guidewire channels are used for passing the one or more guidewires inside the inflatable device,
wherein the guidewire channels are connected to guidewire ports that are attached to one of the shafts of the inflatable device,
wherein the inflatable chambers in the inflation device are the ones that treat the narrowing of fluid-carrying conduits or provide better expansion of blockages in fluid-carrying conduits,
wherein the inflatable chambers are of a non-cylindrical shape or are non-circular in cross-section resulting in the inflatable device having a non-cylindrical shape or a non-circular cross-section,
wherein the inflatable chambers are attached to the shaft of the inflatable device with the walls of the inflatable chambers partially fused to the shaft and connected to the inflation channels, this attachment and connection is air-tight and fluid-tight to limit leakage,
wherein the inflatable device has variable maximum and minimum length, width, and/or depth with each of the one or more inflatable chambers having variable maximum and minimum length, width, and/or depth when inflated and deflated for different sizes of fluid-carrying conduits,
wherein the inflatable chambers are folded around the shaft of the inflatable device when the device is deflated to minimize the overall diameter of the device and to enable insertion of the device in smaller fluid-carrying conduits,
wherein the inflatable chambers achieve better distribution of the force being applied and allow the use of a lower nominal pressure of the inflatable device to reduce acute complications,
wherein the one or more main inflatable chambers are attached to the shafts of the inflatable device and additional one or more side inflatable chambers are associated in a configuration selected from a group of configurations comprising a configuration where the one or more side inflatable chambers are attached to, fused to, or located adjacent to the outer part of the one or more main inflatable chambers, and a configuration where the one or more side inflatable chambers is built into the walls of one of the one or more main inflatable chambers,
wherein the additional side inflatable chambers are associated in a configuration selected from a group of configurations comprising a configuration where the one or more side inflatable chambers have their own, separate inflation channels that run through the shaft of the device, and a configuration where the one or more side inflatable chambers are attached to a shared inflation channel, which is shared with the inflation channel that is connected to one of the one or more main inflatable chambers,
wherein the inflation channels have a configuration selected from a group of configurations comprising a configuration where the inflation channels are connected to their own, separate inflation port, and a configuration where two or more inflation channels have a shared inflation port,
wherein the inflatable chambers have walls that are made of materials selected from a group comprising polyvinyl chloride, polyethylene, polyethylene terephthalate, polyolefin copolymers, nylon derivatives, nylon-reinforced polyurethane, co-extruded copolymers, metallic elements, and alloys,
wherein the walls of the inflatable chambers are formed from one or more layers of the same or different materials to enable shaping of the inflatable device,
wherein the said materials are infused, attached, fused, or impregnated in the walls of the inflatable chamber for added functionality,
wherein the one or more inflatable chambers have a non-cylindrical shape either when inflated, deflated, or both,
wherein the non-cylindrical shape is selected from a group comprising oblong, oval, star-like with rounded edges, and triangular with rounded edges shape as viewed from the X, Y, and/or Z axis of one of the shafts of the device,
wherein the inflatable chambers are angulated either when inflated, deflated, or both, compared to one of the shafts of the device and/or one of the guidewire channels, and the angle is selected from a group ranging between 0°- and 359°-degrees angle,
wherein the inflatable chambers are selected from the group comprising symmetric and asymmetric to one of the shafts of the device and/or one of the guidewire channels along the X, Y, and/or Z axis of one of the shafts of the device leading to the shafts and/or guidewire channels not being in the centre of the inflatable chambers,
wherein the inflatable chambers optionally have convex or concave segments on the exterior parts to enable better application of force when the inflatable chamber is inflated,
wherein the guidewire channels are selected from a group of conformations comprising being on the side of the inflation channels adjected to the wall of the inflatable chambers with zero width between the wall of the inflatable channels and the guidewire channels and being attached to the outside of the inflation channel walls,
wherein the one or more inflatable chambers are inflated in a manner selected from inflation manners selected from a group comprising an inflation manner where the one or more inflatable chambers are inflated at the same pressure with the same inflation agent, and an inflation manner where the one or more inflatable chambers are inflated at different pressures and/or with different inflation agents,
wherein the inflation agent is a gas and/or a liquid,
wherein the inflation channels are optionally connected through air-tight and fluid-tight means to limit leakage to one or more pressure monitors that are connected through inflation hubs to accurately monitor the internal pressure of the inflatable chambers to lower the risk of rupture of the inflatable chambers,
wherein the inflatable device is impregnated or covered with medications to help prevent inflammation, scarring, anticoagulation,
wherein the inflatable device is selected from a group comprising a medical device and a non-medical device,
wherein the medical device is used to treat the narrowing of the fluid-carrying conduits or to treat the expansion of blockages in the fluid-carrying conduits in the body of an animal, wherein the fluid-carrying conduits include blood vessels, cardiac and non-cardiac chambers, and channels in the body of the animal,
wherein the animal is a mammal, and
wherein the non-medical device is used to treat the narrowing of the fluid-carrying conduits or to treat the expansion of blockages in fluid-carrying conduits, wherein the fluid-carrying conduits include a tube, channel, conduit, or duct, wherein the fluid-carrying conduits regulate the flow of a liquid through said tube, channel, conduit, or duct.

12. The inflatable device of claim 11, wherein the one or more protrusions and/or one or more side inflatable chambers are formed by making a molded inflatable chamber with a shaped profile, wherein the shaped profile is a non-cylindrical shape or non-circular in cross-section resulting in the inflatable device having a non-cylindrical shape or a non-circular cross-section, wherein the protrusions and/or the side inflatable chambers have a round, oval, finger-like, irregular or any other shape, wherein the protrusions and/or the side inflatable chambers are selected from a group of configurations where they are similar in size and shape, and they are variable in size and shape, wherein the size and shape variability follows a pattern along the X, Y, and/or Z axis of one of the shafts of the device, and wherein the protrusions and side inflatable chambers have varied maximal and minimal width, length, location, orientation along the X, Y, and/or Z axis of one of the shafts of the device.

13. The inflatable device of claim 11, wherein the one or more protrusions have walls with different physical properties in some parts of the protrusions or the entire protrusions to the main inflatable chamber from the periphery of which they are bulging out of, wherein the different physical properties include thickness, density of the walls of the protrusions, and number of layers that the walls of the protrusions form, wherein the walls of the protrusions have a different number of layers forming the walls to the other parts of the main inflatable chamber from the periphery of which they are bulging out of, wherein the walls of the protrusions have a different composition to the other parts of the main inflatable chamber from the periphery of which they are bulging out of, wherein the one or more protrusions have walls with different mechanical properties in some parts of the protrusions or the entire protrusions to the main inflatable chamber from the periphery of which they are bulging out of, wherein the different physical properties include elasticity, and strength, wherein the protrusions and the main inflatable chamber from the periphery of which they are bulging out of have different expansion properties to allow flexible and independent, semi-flexible and independent, semi-independent, or linked movement between the protrusions and the main inflatable chamber from the periphery of which they are bulging out of owing to the differences in their materials, physical properties, mechanical properties, wherein the said differences allow application of a higher pressure per surface area and/or higher sheer stress to an area of the fluid-carrying conduits when the protrusion in the inflatable device are inflated owing to differences in inflation and expansion between the protrusions and the main inflatable chambers, and at the same time, the said differences minimize the pressure being applied to the other areas of the fluid-carrying conduits, and improve the distribution of force being applied to better dilate the fluid-carrying conduits, wherein the area of the fluid-carrying conduits is a diseased, affected, or targeted area which is to be treated for the narrowing of the fluid-carrying conduits or for the expansion of blockages in the fluid-carrying conduits, and the other areas of the fluid-carrying conduits are normal, unaffected, or non-target areas for narrowing or expansion of the fluid-carrying conduits, wherein the protrusions have a pattern of distribution of their location, size, and/or shape along the perimeter and/or length of the main inflatable chamber from the periphery of which they are bulging out of, wherein the protrusions and the main inflatable chamber from the periphery of which they are bulging out expand in a manner selected from a group comprising a manner where the protrusions and the main inflatable chamber expand at the same speed, and a manner where the protrusions and the main inflatable chamber expand at varying speeds owing to the protrusions being more receptive or more resistant than the other parts of the main inflatable chamber to inflation due to the different physical or mechanical properties of the walls of the protrusions to the other parts of the main inflatable chamber.

14. The inflatable device of claim 11, wherein the one or more side inflatable chambers have walls with different physical properties in some parts of the side inflatable chambers or the entire side inflatable chambers to the main inflatable chamber they are attached to, wherein the different physical properties include thickness, density of the walls of the side inflatable chambers, and number of layers that the walls of the side inflatable chambers form, wherein the walls of the side inflatable chambers have a different number of layers forming the walls to the other parts of the main inflatable chamber they are attached to, wherein the walls of the side inflatable chambers have a different composition to the other parts of the main inflatable chamber they are attached to, wherein the one or more side inflatable chambers have walls with different mechanical properties in some parts of the side inflatable chambers or the entire side inflatable chambers to the main inflatable chamber they are attached to, wherein the different physical properties include elasticity, and strength, wherein the side inflatable chambers and the main inflatable chamber they are attached to have different expansion properties to allow flexible and independent, semi-flexible and independent, semi-independent, or linked movement between the side inflatable chambers and the main inflatable chamber they are attached to owing to the differences in their materials, physical properties, mechanical properties, wherein the side inflatable chambers and the main inflatable chamber to which they are associated expand in a manner selected from a group comprising a manner where the side inflatable chambers and the main inflatable chamber expand at the same speed, and a manner where the side inflatable chambers and the main inflatable chamber expand at varying speeds owing to the side inflatable chambers being more receptive or more resistant than the other parts of the main inflatable chamber to inflation due to the different physical or mechanical properties of the walls of the side inflatable chambers to the other parts of the main inflatable chamber.

15. The inflatable device of claim 11, wherein the inflatable device has a stent or other metallic device mounted on the inflatable part of the device, wherein the stent or the other metallic device can be deployed at the same time as when the inflatable chamber is inflated, or later on after inflating the device.

16. The inflatable device of claim 11, wherein the shafts of the inflatable device, the main inflatable chambers, the side inflatable chambers, and/or the protrusions optionally have one or more radio-opaque markers built, attached, or fused into the walls of the shafts of the inflatable device, the main inflatable chambers, the side inflatable chambers, and/or the protrusions, for defining the orientation of the shafts of the inflatable device, main inflatable chambers, the side inflatable chambers, and/or the protrusions when the inflatable device is rotated and to define the location and extent of the shafts of the inflatable device, main inflatable chambers, the side inflatable chambers, and/or the protrusions, and wherein the shape of the marker is variable.

17. The inflatable device of claim 11, wherein the main inflatable chambers, the side inflatable chambers, and/or the protrusions optionally have segments covered with one or more covering materials, wherein the covering materials comprise metal, metallic blades, and other cutting elements, wherein the covering materials aid the main inflatable chambers, the side inflatable chambers, and/or the protrusions and in turn, the inflatable device form various shapes, and wherein the covering materials are embedded, attached or fused to the segments of the main inflatable chambers, the side inflatable chambers, and/or the protrusions.

18. An inflatable device, the inflatable device comprises:
one or more shafts;
one or more shaft tips;
one or more main inflatable chambers;
one or more side inflatable chambers;
one or more inflation channels;
one or more protrusions;
one or more accessories;
one or more guidewire channels;
one or more guidewire ports;
one or more stiffening wires; and
one or more inflation ports,
wherein the inflatable device is shaped to treat the narrowing of fluid-carrying conduits or to treat the expansion of blockages in fluid-carrying conduits,
wherein the shafts provide the main structure or skeleton of the inflatable device,
wherein the shafts are made from a flexible material,
wherein the shafts are non-inflatable,
wherein the shafts have one or more ports that are connected to the different channels inside the shaft,
wherein the said ports in the shafts are connected to inflation devices, syringes, and manometers to measure pressure,
wherein the one or more inflation channels are located inside one of the shafts of the inflatable device and run along the entire length or part of the said one shaft of the device,
wherein the inflation channels are used for inflating the inflatable chambers,
wherein the inflation channels are air-tight and fluid-tight to limit leakage,
wherein the inflation channels are connected to the inflation ports on one side and the inflatable chambers on the other side,
wherein the protrusions are bulging out of the periphery of one of the one or more main inflatable chambers and the wall of the protrusion is contiguous with the wall of the main inflatable chamber of which it is bulging out to allow inflation at the same time,
wherein the one or more protrusions and the one or more side inflatable chambers are present in a configuration of the inflatable device selected from a group comprising a configuration when the device is deflated, a configuration when the device is inflated, and a configuration when they are present when the device is inflated and deflated,
wherein the one or more accessories include constrictive wires, lines, cylinders, and tubes,
wherein the one or more accessories are selected from a group of configurations where they are made from the same materials entirely, and where they are made from different materials which follow a pattern,
wherein the one or more accessories are made from materials that include non-flexible, semi-flexible material, memory metal, other metals,
wherein the memory metal includes Nitinol,
wherein the other metals include stainless steel, and titanium,
wherein the one or more guidewire channels are located inside one of the shafts of the inflatable device and and run along the entire length or part of the said one shaft of the device,
wherein the guidewire channels are associated with the inflation channels in a configuration selected from a group of configurations comprising a configuration where the guide wire channels run in the center of the shaft to the tip of the inflatable device, with the inflation channels surrounding the guide wire channels, and a configuration where the guide wire channels run parallel and/or adjacent to the inflation channels inside the shafts of the inflatable device,
wherein the guidewire channels are used for passing the one or more guidewires inside the inflatable device,
wherein the guidewire channels are connected to guidewire ports that are attached to one of the shafts of the inflatable device,
wherein the inflatable chambers in the inflation device are the ones that treat the narrowing of fluid-carrying conduits or provide better expansion of blockages in fluid-carrying conduits,
wherein the inflatable chambers are of a non-cylindrical shape or are non-circular in cross-section resulting in the inflatable device having a non-cylindrical shape or a non-circular cross-section,
wherein the inflatable chambers are attached to the shaft of the inflatable device with the walls of the inflatable chambers partially fused to the shaft and connected to the inflation channels, this attachment and connection is air-tight and fluid-tight to limit leakage,
wherein the inflatable device has variable maximum and minimum length, width, and/or depth with each of the one or more inflatable chambers having variable maximum and minimum length, width, and/or depth when inflated and deflated for different sizes of fluid-carrying conduits,
wherein the inflatable chambers are folded around the shaft of the inflatable device when the device is deflated to minimize the overall diameter of the device and to enable insertion of the device in smaller fluid-carrying conduits,
wherein the inflatable chambers achieve better distribution of the force being applied and allow the use of a lower nominal pressure of the inflatable device to reduce acute complications,
wherein the inflatable chambers have walls that are made of materials selected from a group comprising polyvinyl chloride, polyethylene, polyethylene terephthalate, polyolefin copolymers, nylon derivatives, nylon-reinforced polyurethane, co-extruded copolymers, metallic elements, and alloys,
wherein the walls of the inflatable chambers are formed from one or more layers of the same or different materials to enable shaping of the inflatable device,
wherein the said materials are infused, attached, fused, or impregnated in the walls of the inflatable chamber for added functionality,
wherein the one or more inflatable chambers have a non-cylindrical shape either when inflated, deflated, or both,
wherein the non-cylindrical shape is selected from a group comprising oblong, oval, star-like with rounded edges, and triangular with rounded edges shape as viewed from the X, Y, and/or Z axis of one of the shafts of the device,
wherein the inflatable chambers are angulated either when inflated, deflated, or both, compared to one of the shafts of the device and/or one of the guidewire channels, and the angle is selected from a group ranging between 0°- and 359°-degrees angle,
wherein the inflatable chambers are selected from the group comprising symmetric and asymmetric to one of the shafts of the device and/or one of the guidewire channels along the X, Y, and/or Z axis of one of the shafts of the device leading to the shafts and/or guidewire channels not being in the centre of the inflatable chambers,
wherein the inflatable chambers optionally have convex or concave segments on the exterior parts to enable better application of force when the inflatable chamber is inflated,
wherein the guidewire channels are selected from a group of conformations comprising being on the side of the inflation channels adjected to the wall of the inflatable chambers with zero width between the wall of the inflatable channels and the guidewire channels and being attached to the outside of the inflation channel walls,
wherein the one or more inflatable chambers are inflated in a manner selected from inflation manners selected from a group comprising an inflation manner where the one or more inflatable chambers are inflated at the same pressure with the same inflation agent, and an inflation manner where the one or more inflatable chambers are inflated at different pressures and/or with different inflation agents,
wherein the inflation agent is a gas and/or a liquid,
wherein the inflation channels are optionally connected through air-tight and fluid-tight means to limit leakage to one or more pressure monitors that are connected through inflation hubs to accurately monitor the internal pressure of the inflatable chambers to lower the risk of rupture of the inflatable chambers,
wherein the inflatable device is impregnated or covered with medications to help prevent inflammation, scarring, anticoagulation,
wherein the inflatable device is selected from a group comprising a medical device and a non-medical device,
wherein the medical device is used to treat the narrowing of the fluid-carrying conduits or to treat the expansion of blockages in the fluid-carrying conduits in the body of an animal, wherein the fluid-carrying conduits include blood vessels, cardiac and non-cardiac chambers, and channels in the body of the animal,
wherein the animal is a mammal, and
wherein the non-medical device is used to treat the narrowing of the fluid-carrying conduits or to treat the expansion of blockages in fluid-carrying conduits, wherein the fluid-carrying conduits include a tube, channel, conduit, or duct, wherein the fluid-carrying conduits regulate the flow of a liquid through said tube, channel, conduit, or duct.

19. The inflatable device of claim 18, wherein the one or more accessories are associated with the inflatable device in a manner selected from configurations where they are embedded, fused, and/or attached to the walls of the main inflatable chamber and/or the walls of the side inflatable chambers walls, and/or the walls of the protrusions, and where they are located on the outside of the walls of the main inflatable chamber and/or the walls of the side inflatable chambers walls, and/or the walls of the protrusions, and wherein the one or more accessories are associated with the inflatable device in the manner that upon inflation of the inflatable device, the presence of the one or more accessories results in restriction of the expansion of the main inflatable chamber and/or the side inflatable chambers, and/or the protrusions, wherein the restriction in expansion is in a pattern that creates a shaped inflatable device required to treat the narrowing of the fluid-carrying conduits or to treat the expansion of blockages in fluid-carrying conduits, wherein the pattern includes configurations selected from a group comprising a configuration where the one or more accessories are located at one or more locations along the X, Y, and/or Z axis of the shafts of inflatable device, and/or a configuration where the one or more accessories are distributed at and around one or more parts of the main inflatable chamber and/or the side inflatable chambers, and/or the protrusions, wherein said configurations are used to create the shaped inflatable device required to treat the narrowing of the fluid-carrying conduits or to treat the expansion of blockages in fluid-carrying conduits.

20. The inflatable device of claim 18, wherein the inflatable device has a stent or other metallic device mounted on the inflatable part of the device, wherein the stent or the other metallic device can be deployed at the same time as when the inflatable chamber is inflated, or later on after inflating the device.
